(19) United States
(12) Patent Application Publication
(10) Pub. No.: US 2009/0012261 A1
(43) Pub. Date: Jan. 8, 2009

(54) HLA Epitope Identification

(76) Inventors: Ma Luo, Winnipeg (CA); Frank Plummer, Winnipeg (CA); Terry Blake Ball, Winnipeg (CA); Christina Semeniuk, Winnipeg (CA); Harold Peters, Winnipeg (CA); Rupert Capina, Winnipeg (CA); Mark Mendoza, Winnipeg (CA)

Correspondence Address: ADE & COMPANY INC., 2157 Henderson Highway, WINNIPEG, MB, R2G1P9, CA

(21) Appl. No.: 12/051,543

(22) Filed: Mar. 19, 2008

Related U.S. Application Data
(60) Provisional application No. 60/895,607, filed on Mar. 19, 2007.

(51) Int. Cl. C07K 7/00

(52) U.S. Cl. 530/326; 530/328

(57) ABSTRACT

The present invention describes ways to identify HLA allele-specific epitopes that result from HLA restriction of antigen-specific cellular immune responses. The invention employs a combination of bioinformatics and functional assays to systematically identify and classify CTL mutations to determine correlates of virus-host interactions Peptides representing HLA allele-specific epitopes are provided as well as methods for validating HLA-restricted epitopes and methods for measuring T cell responses.

### PRIOR APPLICATION INFORMATION

This application claims the benefit of U.S. Provisional Patent Application 60/895,607, filed Mar. 19, 2007.

### FIELD OF INVENTION

The present invention describes how to identify HLA allele-specific epitopes that result from HLA restriction of antigen-specific cellular immunity. A combination of bioinformatics and functional assays is used to identify pathogen mutations relevant to CTL responses.

### BACKGROUND OF THE INVENTION

HLA is one of the most important factors that is directly involved in immune responses to pathogens, e.g. HIV-1, and contribute to the variations in response to infection and disease. Identifying the associations of HLA alleles with different infection and disease outcomes and clarifying which epitopes can they bind and present to the CTLs are useful for developing T cell based vaccines to pathogens such as HIV-1 and to develop diagnostic tools to monitor effective immune responses for the vaccine trial.

Traditional methods for epitope identification include algorithmic identification, peptide elution and cell-based binding assay techniques. The success and speed of these traditional approaches has been impeded by limited amount of patient PBMCs, laborious lab work, low throughput, poor reproducibility or restriction to only the most commonly studied MHC types.

### SUMMARY OF THE INVENTION

In describing the present invention, HIV is used throughout to illustrate how the invention may be employed and how the results revealed are useful in developing further research tools, diagnostics and therapeutics. It should be understood that HIV is only an exemplary pathogen. The methods and compositions described herein may be applied to a wide range of microorganisms in relation to the immune responses they elicit.

Thus the present invention is based on, but is not limited to, our initial identification of positively selected amino acids in individual proteins of gag. We analyzed more than 1000 proviral gag sequences from the HIV positive women of the sexworkers cohort using QUASI analysis, correlating the positive selection sites with patient HLA type, and further classifying the selected variants as beneficial or detrimental to the virus by correlation with the patients' CD4 counts. We confirmed that the potential epitopes identified through this approach were actual epitopes by ELISPOT assays with peptides overlapping the identified region using patient PBMCs. Using this combination of bioinformatics and functional assay, we can identify new HLA epitopes.

In one aspect, the invention describes methods for identifying HLA allele-specific sequence variations in a target microorganism, e.g. HIV, hepatitis virus, herpes virus, that result from HLA restriction of antigen-specific cellular immune responses.

In another aspect, the invention describes a method for identifying an HLA Class I epitope, the method comprising the steps of: (a) analyzing an amino acid sequence encoded by a pathogen isolated from subjects infected with the pathogen, to identify positively or negatively selected amino acids; (b) correlating the presence of the positive/negatively selected amino acids identified in step (a) with HLA Class I alleles typed from the subjects, to identify which selected amino acids occur in association with which HLA Class I allele, thereby identifying potential epitopes for the associated HLA Class I allele, wherein the potential epitopes are defined by peptide sequences which contain the positively selected amino acids; and (c) validating that the potential epitope from step (b) is an actual epitope recognized by the associated HLA Class I allele. The validating step comprises the steps of: (i) synthesizing peptides, typically consisting of 7 to 15 residues, usually from 8 to 12 residues and more usually from 9 to 11 residues, and containing the positive selected amino acid from step (b) at an anchor position, the peptides having, in the remaining amino acid positions, the sequence corresponding to the amino acid sequence encoded by the pathogen; and (ii) testing the peptides to determine if the peptides bind to the associated HLA Class I molecule; wherein binding between the peptide and the associated HLA Class I molecule indicates that the peptide is a peptide ligand (epitope) of the associated HLA Class I molecule.

The analysis of amino acid sequences encoded by a pathogen isolated from infected subjects and the correlation between the positive (or negative) selected amino acids with HLA alleles typed from the subjects to identify potential epitopes for the associated ELA Class I allele may be achieved by methods which determine the influence of variation in host genes on selection of microorganisms having amino acid substitutions. Such a determination may comprise (i) selecting a population of subjects infected with the microorganism of interest and typing all individuals of the cohort for at least one selected HLA allele involved in the host's response to the microorganism; (ii) determining at least part of a polynucleotide or polypeptide sequence in the microorganism in a statistically sufficient number of subjects from each type identified in step (i) in the cohort; (iii) determining the consensus (i.e. most common) amino acid across the cohort at each position of the sequence analysed in step (ii); (iv) comparing the results of step (i) with the results of step (ii) to determine whether the subjects' HLA allele in step (i) increases or decreases the probability of a sequence variation in the microorganism at the first amino acid of the sequence determined in step (ii); and (v) repeating step (iv) for each amino acid of the sequence identified in step (ii).

In various embodiments, positively selected amino acids are preferred.

In some embodiments, the peptide whose HLA restriction is to be validated may contain the positively selected amino acid at anchor position 2, 8 or 9. The potential epitopes containing the positively selected amino acids typically consist of 7 to 15 residues, usually from 8 to 12 residues and more usually from 9 to 11 residues, e.g. at step (b). Step (a) may be performed using the algorithm employed in the software QUASI.

Testing a peptide to determine its HLA-specificity may be performed using methods known in the art. One method is simply to assay for binding of the peptide to the associated HLA Class I molecule. Another method is to test the peptide for HLA Class I allele-specific T-cell stimulation, e.g. using the ELISPOT assay described below.

The pathogen may be a virus, especially those whose genomes evolve significantly to escape the HLA-specific selective pressures of the host as indicated by their sequence variations (polymorphisms), e.g. human immunodeficiency virus (HIV), herpes viruses and hepatitis viruses including hepatitis C.

In the context of HIV, the methods described herein can apply specifically to subjects who are long term non-progressors (LTNPs). Specifically, the amino acid sequences under analysis include those encoded by HIV-1 such as the P7-P1 region of HIV-1 defined by the sequence RQANFLGKIWPSSKGRPGNF (SEQ ID No. 1). The amino acid sequences under analysis also include other regions of gag, such as P17, p24, p2 and p6 (FIG. 3).

In another aspect, the invention provides synthesized peptides overlapping P7-P1 region, some of which are the epitopes of HLA Class I alleles. Thus the invention provides an isolated peptide which is an HLA Class I peptide ligand. In particular, certain peptides are T cell epitopes from the P7-P1 region of HIV-1, said P7-P1 region being defined by the sequence RQANFLGKIWPSSKGRPGNF (SEQ ID No. 1).

The invention also provides a peptide region, which contains HLA Class I epitopes from the P7-P1 region of HIV-1, and also provides peptide ligands whose sequences are the epitope sequences from the P7-P1 region, the P7-P1 region being defined by the sequence RQANFLGKIWPSSKGRPGNF (SEQ ID No. 1), the peptide being identified by the method described above. In some embodiments, the peptide defines the linear length encompassed by a T cell epitope, typically 7 to 15 residues, usually from 8 to 12 residues and more usually from 9 to 11 residues. In some embodiments, the peptides represent peptide ligands of HLA Class I B*1302. In some embodiments, the peptide comprises a sequence selected from the group consisting of (i) RQANFLGKI (SEQ ID No. 2), (ii) RQANFLGRI (SEQ ID No. 3), (iii) KIWPSSKGR (SEQ ID No. 4), (iv) KLWPSNKGR (SEQ ID No. 5), (v) SNKGRPGNF (SEQ ID No. 6), (vi) SSKGRPGNF (SEQ ID No. 7), and (vii) LGKIWPSSK (SEQ ID No. 8).

In some embodiments, the peptide is from the P7-P1 region of HIV-1, said P7-P1 region being defined by the sequence RQANFLGKIWPSSKGRPGNF, the peptide representing epitopes of HLA Class I A*7401 and comprising a sequence selected from the group consisting of (i) SNKGRPGNF (SEQ ID No. 6), (ii) LGKIWPSSK (SEQ ID No. 8), (iii) GKIWPSSKG (SEQ ID No. 9), (iv) SSKGRPGNF (SEQ ID No. 7), (v) GRIWPSNKG (SEQ ID No. 10), (vi) LGKIWSSNK (SEQ ID No. 11), (vii) RQANFLGKI (SEQ ID No. 12) and (viii) RQANFLGRI (SEQ ID No. 3). We contemplate the peptides (i) SNKGRPGNF (SEQ ID No. 6), (ii) LGKIWPSSK (SEQ ID No. 8), (iii) GKIWPSSKG (SEQ ID No. 9) as high-affinity epitopes for A*7401, whereas the peptides (iv) SSKGRPGNF (SEQ ID No. 7), (v) GRIWPSNKG (SEQ ID No. 10), (vi) LGKIWSSNK (SEQ ID No. 11), (vii) RQANFLGKI (SEQ ID No. 12) and (viii) RQANFLGRI (SEQ ID No. 3) could be low affinity epitopes for A*7401.

In some embodiments, the peptides are from the gag region of HIV-1, said gag region being defined by the sequence in FIG. 3, the peptides representing epitopes of various HLA Class I alleles and comprising the peptide ligand sequences identified in Table 4.

Another aspect of the present invention is a method of preparing a composition comprising making either an amino acid sequence designed according to the above methods or a vector construct capable of expressing that sequence in a patient, which is able to inducing a specific T-cell response in a patient infected with a micro-organism or at risk of infection with that microorganism, and then combining the therapeutic with a pharmaceutically acceptable excipient.

The invention also describes compositions for inducing a T-cell response to HIV, the compositions comprising either the peptide identified by the above methods or a vector construct capable of expressing the peptide sequence in a subject. Such peptides and constructs should be useful for inducing a specific T-cell response in an HIV-infected subject or in a subject at risk of HIV infection. The composition may include a pharmaceutically acceptable excipient and/or a carrier such as physiologic saline, and/or an adjuvant.

The invention also describes methods for inducing a T cell response against an antigen by administering to a subject either the peptide identified by the above methods or a vector construct capable of expressing the peptide sequence in the subject to induce a specific T-cell response. The cellular response may be a CD8+ T cell response, a CD4+ T cell, or both a CD8+ T cell and a CD4+ T cell response.

In another aspect, the invention describes a method for validating a test peptide as a peptide ligand (epitope) of HLA Class I, the method comprising (a) contacting the peptide identified as described above with an HLA Class I molecule and detecting binding, thereby defining the level of binding; and (b) contacting the test peptide with the HLA Class I molecule and detecting binding; wherein the level of binding in step (b) relative to step (a) determines whether the test peptide is a peptide ligand of the HLA Class I molecule. The method may relate to the HLA Class I molecule B*1302, or A*7401 or the HLA alleles listed in Table 4 wherein the binding in step (a) defines the positive control level of binding.

In another aspect, the invention provides an isolated complex comprising the peptide defined as above, bound to a HLA Class I molecule. In specific embodiments, the complex is a tetramer of HLA Class I (e.g. B*1302, A*7401, and alleles listed in Table 4) bound to the peptide. In the tetrameric complex, the HLA Class I molecules may be conjugated to avidin to form the tetramer. The complex may further comprise a detectable label.

In another aspect, the invention describes a method for determining the HLA Class I specificity of the peptide described above, the method comprising the step of contacting the peptide with an HLA Class I molecule and determining whether the peptide binds to the HLA Class I molecule.

In another aspect, the invention describes a method for measuring T cell response, the method comprising the steps of: (a) obtaining from a subject a blood sample containing T cells; (b) producing a complex comprising the peptide as described above, bound to a HLA Class I molecule; (c) contacting the T cells from step (a) with the complex from step (b); and (d) measuring binding between the complex and the T cell; wherein the level of binding in step (c) is a measure of T cell response to the peptide in the subject. In certain embodiments, the complex may further comprise a detectable label.

In another aspect, the invention describes a method for measuring T cell response, the method comprising the steps of: (a) obtaining from a subject a blood sample containing T cells; (b) contacting the T cells with the complex comprising the peptide as described above; and (c) measuring binding between the complex and the T cell; wherein the level of binding in step (c) is a measure of T cell response to the peptide in the subject.

These and other aspects of the present invention are more fully described having regard to the following drawings and detailed description. The drawings and description are provided to aid in the description of the invention but should not be regarded as a limiting aspect of the invention.

### BRIEF DESCRIPTION OF THE DRAWINGS

The present invention will be further understood from the following description with reference to the drawings, in which:

FIG. 1 shows the consensus peptides designed to overlap by 8 amino acid residues spanning the entire p1 region and part of the p7 region.

FIG. 2 shows the two consensus and two mutated peptides that would be tested for positive selection at position 5 of p1, ensuring that the positive selected amino acids are at position 2 or 8 of the peptide.

FIG. 3 is a map of positively selected mutations across gag generated by QUASI for clade A1 and D viruses. Consensus sequence is shown as single line of residues with positively selected (upper case) and neutral selected (lower case) shown underneath the consensus for each site. Predicted proteasomal cleavage sites of the consensus sequence are shown as asterisks (*) at an IC50 threshold score of 0.5. Mutations that affect proteasomal cleavage are shown in red, and those cleavage sites that are abolished through mutations are shown as red asterisks. Mutations that affect predicted TAP transport are shown in green; mutations that affect N-terminal trimming are shown in pink; mutations that may result in reduction of HLA binding are shown in blue; and mutations that may reduce TCR recognition are shown in yellow. Mutations that correlate with differences in CD4 counts are boxed.

FIG. 4 (A) Identified positively selected amino acids in the p1 spacer protein (underlined); (B) Their position in the p1 stem loop structure.

FIG. 5 Peptide response for pool 7.

FIG. 6 Peptide response for pool B.

FIG. 8 Peptide response for pool 14.

FIG. 9 Peptide response for pool 15.

FIG. 10 Peptide response for pool 30.

FIG. 11 Peptide response for pool 31.

FIG. 12 Peptide response for pool 32.

FIG. 13 Peptide response for pool 33.

FIG. 14 Peptide response for pool 40.

FIG. 15 Peptide response for pool 41.

FIG. 16 Peptide response for pool 45.

FIG. 17 Peptide response for pool 56.

FIG. 18 Peptide response for pool 58.

FIG. 19 Peptide response for pool 59.

### DETAILED DESCRIPTION OF EMBODIMENTS

The present invention is based on, but is not limited to, our initial identification of positively selected amino acids in individual proteins of gag. This work, described herein only as a working embodiment from which the invention is derived, analyzed more than 1000 proviral gag sequences from the HIV positive women of the sexworkers cohort using QUASI analysis. The positive selection sites were then correlated with patient HLA type to identify potential epitopes by statistical analysis, and further classified as beneficial or detrimental to the virus by correlation with the patients' CD4 counts. This allows us to tease apart the positively selected amino acids that arose and affect each step of peptide presentation and classify HLA restricted immune responses that are either beneficial or detrimental to the virus. The information can be used in treatment of disease by enhancing immune responses that detrimental to the virus and preventing immune responses that associated with lower CD4 counts. The potential epitopes that were identified through this approach were confirmed as HLA-restricted peptide ligands by ELISPOT assays with peptides overlapping the identified region using patient PBMCs.

Using this approach we have identified many potential HLA epitopes that have not been identified by traditional approach. First round of ELISPOT analysis for peptides spanning p1 and part of p7 of gag has confirmed the identified potential epitopes are indeed recognized by patients carrying the correlated HLA alleles.

We also correlated the positively selected amino acids with the predicted proteasomal cleavage sites (NetChop 3.0) and transport efficiency (TAP affinity algorithm). However, it is noted that such positively selected amino acid correlated with predicted proteasomal cleavage sites and transport efficiency tend to indicate regions of the pathogenic sequence that lie outside the CTL epitopes, i.e. certain HLA-associated sequence variations in a pathogen may indicate viral escape by disruption of proteasome peptide cleavage or disruption of some other peptide processing mechanism. Since such sequence variations are not CTL epitopes (i.e. not ELA-binding peptide ligands), HLA-binding assays should not identify these regions as epitope sequences.

The invention describes a method for identifying an HLA Class I epitope, the method comprising the steps of: (a) analyzing an amino acid sequence encoded by a pathogen isolated from subjects infected with the pathogen, to identify positively or negatively selected amino acids (preferably positively selected); (b) correlating the presence of the positive/negatively selected amino acids identified in step (a) with HLA Class I alleles typed from the subjects, to identify which positively selected amino acids occur in association with which HLA Class I allele, thereby identifying potential epitopes for the associated HLA Class I allele, wherein the potential epitopes are defined by peptide sequences which contain the positively selected amino acids; and (c) validating that the potential epitope from step (b) is an actual epitope recognized by the associated HLA Class I allele. The validating step comprises the steps of: (i) synthesizing a peptide consisting of 7 to 15 residues, usually from 3 to 12 residues and more usually from 9 to 11 residues, and containing the positive selected amino acid from step (b) at an anchor position, the peptide having, in the remaining amino acid positions, the sequence corresponding to the amino acid sequence encoded by the pathogen; and (ii) testing the peptide to determine if the peptide binds to the associated HLA Class I molecule; wherein binding between the peptide and the associated HLA Class I molecule indicates that the peptide is a peptide ligand of the associated HLA Class I molecule.

The above method includes analysis of amino acid sequences encoded by a pathogen isolated from infected subjects and the correlation between the positive (or negative) selected amino acids with HLA alleles typed from the subjects to identify potential epitopes for the associated HLA Class I allele. Such a correlative analysis may be achieved by methods, which determine the influence of variation in host genes on selection of microorganisms having amino acid substitutions. The method comprises (i) selecting a population of subjects infected with the microorganism of interest and typing all individuals of the cohort for at least one selected HLA allele involved in the host's response to the microorganism; (ii) determining at least part of a polynucleotide or polypeptide sequence in the microorganism in a statistically sufficient number of subjects from each type identified in step (i) in the cohort; (iii) determining the consensus (i.e. most common) amino acid across the cohort at each position of the sequence analysed in step (ii), the consensus here being the presumptive wild type sequence; (iv) comparing the results of step (i) with the results of step (ii) to determine whether the subjects' HLA allele in step (i) increases or decreases the probability of a sequence variation in the microorganism at the first amino acid of the sequence determined in step (ii); and (v) repeating step (iv) for each amino acid of the sequence identified in step (ii).

The main anchor positions may be defined as those associated with at least a 10-fold reduction in binding capacity to its associated MHC Class I molecule for the majority of analogues where the putative anchor position is substituted. A secondary anchor position may be defined as one where at least a 3-fold reduction in binding is recorded for over 50% of the substitutions.

The binding of the peptide to the MHC complex is obviously important for identifying potential peptide ligands. It is estimated that only one in about 200 peptides will bind to a given HLA allele (or MHC allele). A very large number of different HLA alleles or MHC alleles exist each with a highly selective peptide binding specificity. The binding motif for a given MHC class I allele is generally, but not always, 8 to 10 amino acids long. The motif is characterized by a strong amino acid preference at specific positions in the motif, usually two or more highly conserved amino acids. These positions are “anchor positions”. For many MHC class I alleles the anchor position are placed at P2 and P9 in the motif. However this is not always the case. Large number of peptide data exist describing this MHC specificity variation. One important source of data is the SYFPEITHI MHC database (56) specifically incorporated herein by reference, which contains information on MHC ligands and binding motifs.

In some embodiments, the peptide e.g. of step (i) above may contain the positively selected amino acid at anchor position 2, 8 or 9. The potential epitopes containing the positively selected amino acids may consist of 7 to 15 residues, usually from 8 to 12 residues and more usually from 9 to 11 residues, e.g. at step (b). Step (a) may be performed using the algorithm employed in the software QUASI.

An algorithm for mapping positively selected members of quasi species-type viruses (QUASI) is described in Stewart et al. 2001 BMC Bioinformatics 2:1 (39). An overabundance of replacement mutations relative to silent mutations in viral proteins serves as a direct indication of the selective advantage gained through variation. QUASI's selection mapping algorithm tests each observed replacement mutation at each codon to identify those particular replacement mutations that are overabundant relative to silent mutations at that codon. Such replacement mutations are determined to be positively selected. Negatively selected variants are recognized as “noise” and are thereafter ignored.

The overall analysis involves HLA-typing of subjects and sequencing the genes encoding the potential pathogenic targets (for example HIV gag proteins). The positive and negative associations between the HLA alleles and the pathogen's sequence variations are determined in a large population of infected subjects. The population should be the same or similar to the population from which the subject was drawn. The pathogen's amino acids that have known associations with the HLA alleles in the subject are then examined.

Such an analysis can reveal regions of amino acid sequence that are either susceptible or resistant to variation. Amino acid residues resistant to change are more likely to have critical structural, catalytic or functional properties since the pathogen must maintain functional integrity. Within these functional constraints, on the basis of associations between host and pathogen sequence variations, one may identify regions in the pathogen that may have been selectively modified to evade influence of the host's immunological response; i.e., the identified regions may represent HLA-restricted CTL-related epitopes. Such an HLA-driven mutation occurs not randomly but driven by the HLA type of the host, enabling a pathogen to avoid or “escape” that host's immune system. For example, if a subject has an A*03 HLA type, the pathogen will favour mutations that avoid A*03 epitopes. Correlations between point-wise (single site) mutations in the HIV sequence and the HLA types of infected subjects can be used to pinpoint the epitopes of the associated HLA alleles.

The HLA allele is indicated in association with certain variations in the pathogen. The association is defined by statistical analysis. By “associated” is meant either directly or indirectly involved in the host's response to the microorganism. For example, the HLA type marker may be HLA Class I (A, B, or C) or HLA Class II (DR, DQ). The association may be negative or positive. For example, change from consensus amino acid at certain positions in HIV p17 may be positively associated with the presence of certain HLA alleles. This means that subjects having those HLA type were significantly more likely to vary from the consensus at these sites compared with all subjects in the cohort that do not have those HLA type. A similar definition applies to negative associations.

HLA typing and/or for identifying sequence variations in the pathogen sequence may be accomplished by a variety of different methods. Some commonly used methods in the art include, for example, microcytotoxicity/serology, direct sequence based typing, sequence specific priming (SSP), sequence specific oligonucleotide (SSO), reverse sequence specific oligonucleotide (reverse SSO), restriction length fragment polymorphism (RFLP) or amplification length fragment polymorphism (AFLP) typing. HLA subtype determination may also be accomplished by a variety of methods known in the art, which include for example, serological or cellular HLA typing techniques as well as non-serological, DNA based HLA typing methods. Non-serological typing methods are preferentially used in the method of the invention. Such methods include, for example: restriction fragment length polymorphism analysis, sequence specific oligonucleotide probing and/or priming techniques, and DNA sequencing. The polymerase chain reaction (PCR) process may also be used to amplify genomic DNA permitting the usage of more convenient HLA typing procedures. In one form of the invention extended Major Histocompatibility Complex (MHC) typing is carried out using sequence analysis: the specific HLA class I genes are amplified by PCR, and sequenced using BigDye cycle sequencing method and typed using a taxonomy-based sequence analysis method.

Methods described herein may be applied to a wide range of pathogenic organisms. Such organisms include but are not limited to bacteria, fungi, mycobacterium, viruses and virus-like particles. The methods described herein have particular value for microorganisms that evolve rapidly to escape the HLA-specific selective pressures of the host as indicated by their sequence variations (polymorphisms). Examples of such organisms include human immunodeficiency virus (HIV) and AIDS related viruses, herpes viruses and hepatitis viruses including hepatitis C and HBV.

Identifying and determining a portion of a polynucleotide and/or polypeptide sequence may be done by any means known in the art. If only the polynucleotide sequence is known, the polypeptide sequence may be predicted or directly sequenced as required.

To determine whether the sequence variations (polymorphisms) in the cohort are distributed randomly or are associated with HLA alleles as a result of selective pressure, the population consensus sequence may be used as a reference sequence and is determined by assigning the most common amino acid in the population at each position. Alternatively, and depending on the analysis being performed, consensus sequences resulted from published sequences in the database [e.g. Los Alamos HIV database, managed and operated by Los Alamos National Security, LLC, Los Alamos Research Park, 4200 West Jemez Road, Suite 200B, Los Alamos, N. Mex. 87544] can be used as the reference sequence. Generally the outcome assessed is any change in the amino acid (even a low but detectable level of mutated or variant sequence) from the consensus sequence of the microorganism being examined. Alternatively, the analysis may be limited to examination of a specific or characteristic amino acid change at a particular residue.

Since antigen specific CTL responses are HLA class I restricted, sequence variations in the pathogen sequence resulting from CTL escape mutation are analyzed to determine whether they are HLA class I allele-specific across the population and would be in residues within or proximate to CTL epitopes. Thus sequence variations within and proximate to known and putative CTL epitopes may be HLA Class I allele specific. For each HLA and positive selected amino acid association, it can be assumed that there is at least one epitope in the vicinity of that association. To look for the most likely epitope that can be recognized by that HLA type, one can search for each 7 to 15 residues, usually from 8 to 12 residues and more usually from 9 to 11 residues long amino-acid sequence in the vicinity of the association. As an example, a search can be conducted in about a 33-long window on either side of the association position. Since the positions flanking an epitope can influence whether it is presented on a cell's surface, the 33-long window allows for a 12-amino-acid-long flanking region on either side of a 9-amino-acid-long epitope.

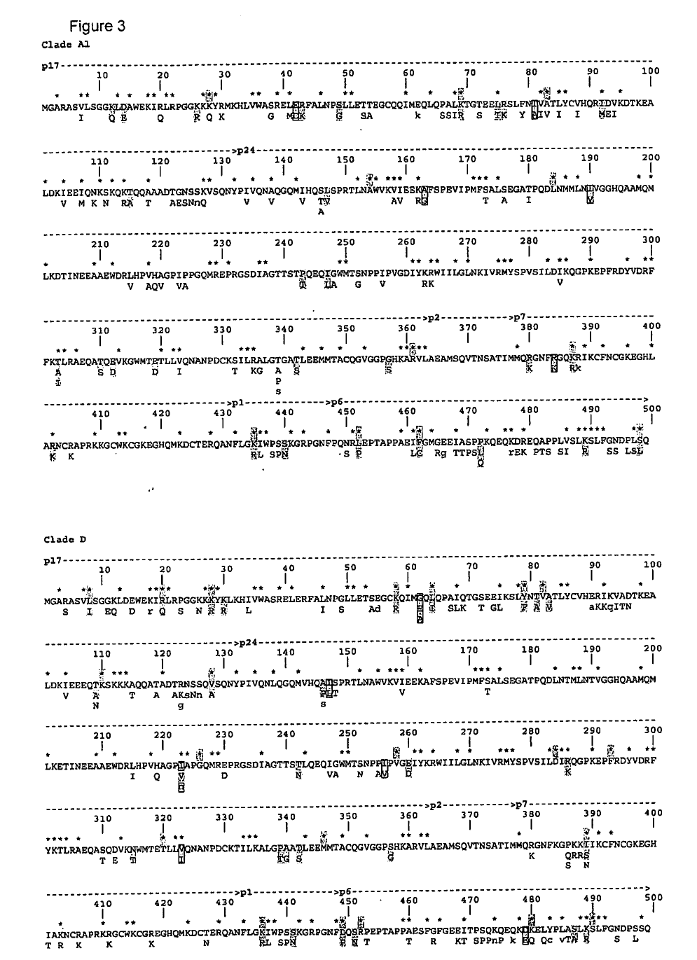

In the context of HIV, the methods described herein can apply specifically to subjects who are long term non-progressors (LTNPs) and HLA alleles that are statistically significantly associated with LTNPs. Specifically, the amino acid sequences under analysis include those encoded by HIV-1 such as the P7-P1 region of HIV-1 defined by the sequence RQANFLGKIWPSSKGRPGNF (SEQ ID No. 1) and the gag sequences in FIG. 3.

In another aspect, the invention describes a method for determining the HLA Class I specificity of the peptide described above, the method comprising the step of contacting the peptide with an HLA Class I molecule and determining whether the peptide binds to the HLA Class I molecule.

In another aspect, the invention describes a method for measuring T cell response, the method comprising the steps of: (a) obtaining from a subject a blood sample containing T cells; (b) producing a complex comprising the peptide as described above, bound to a HLA Class I molecule; (c) contacting the T cells from step (a) with the complex from step (b); and (d) measuring binding between the complex and the T cell; wherein the level of binding in step (c) is a measure of T cell response to the peptide in the subject. In certain embodiments, the complex may further comprise a detectable label.

In another aspect, the invention describes a method for measuring T cell response, the method comprising the steps of: (a) obtaining from a subject a blood sample containing T cells; (b) contacting the T cells with the complex comprising the peptide as described above; and (c) measuring binding between the complex and the T cell; wherein the level of binding in step (c) is a measure of T cell response to the peptide in the subject.

Testing a peptide to determine its HLA-specificity may be performed using methods known in the art. One method is simply to assay for binding of the peptide to the associated HLA Class I molecule. One such assay is the iTopia system (see below). Another method is to test the peptide for HLA Class I allele-specific T-cell stimulation, e.g. using the ELISPOT assay (78). ELISPOT is an immunological assay based on ELISA (Enzyme-Linked Immunosorbent Assay). In ELISA, the substance containing the “unknown” is stuck at the bottom of the well, whereas in ELISPOT the substance with the “unknown” (in this case, peptides) is placed in the well after the bottom of the well has been coated with cytokine-specific antibody. In both cases, the wells are typically contained within a generic microtiter plate. The ELISPOT method is often used to determine the amount (i.e. the concentration) of activated antigen-specific cytotoxic T-cells in a given sample of splenocytes.

ELISPOTs rely on the principle that T cells secrete cytokines following activation. In this assay, a number (usually around 10⁶) splenocytes or peripheral blood lymphocytes are plated in a 96-well nitrocellulose plate with antigen. The T cells settle to the bottom of the plate and, if they are specific for the given antigen or peptide, they will become activated. Because the plates are pre-coated with antibodies to the cytokine of interest, cytokines secreted by activated T cells will be “captured” locally. Typically, CD4 responses are measured by Interleukin-4 capture, while CD8 responses are measured by Ifn-γ (Interferon-gamma) capture.

Following incubation, the T cells can be washed away and a secondary antibody to the same cytokine can be added. This secondary antibody is usually labeled, e.g. biotinylated, and can be visualized by, say, adding streptavidin-alkaline phosphatase reagent. This reagent catalyses the conversion of a substrate to a deep purple stain, causing purple spots to appear wherever an activated T cell was. By counting these spots, we can determine what fraction of T cells can be activated by a given antigen.

In another aspect, the invention provides an isolated peptide which is an HLA Class I-restricted peptide ligand. In particular, certain peptides represent T cell epitopes from the P7-P1 region of HIV-1, said P7-P1 region being defined by the sequence RQANFLGKIWPSSKGRPGNF (SEQ ID No. 1).

The invention also provides an isolated peptide which is an HLA Class I epitope from the P7-P1 region of HIV-1, the P7-P1 region being defined by the sequence RQANFLGKIWPSSKGRPGNF (SEQ ID No. 1), the peptide being identified by the method described above. In some embodiments, the peptide defines the linear length encompassed by a T cell epitope, typically 7 to 15 residues, usually from 8 to 12 residues and more usually from 9 to 11 residues. In some embodiments, the peptides represent peptide ligands of HLA Class I B*1302.

In some embodiments, the peptide comprises a sequence selected from the group consisting of (i) RQANFLGKI (SEQ ID No. 2), (ii) RQANFLGRI (SEQ ID No. 3), (iii) KIWPSSKGR (SEQ ID No. 4), (iv) KLWPSNKGR (SEQ ID No. 5), (v) SNKGRPGNF (SEQ ID No. 6), (vi) SSKGRPGNF (SEQ ID No. 7), and (vii) LGKIWPSSK (SEQ ID No. 8).

In some embodiments, the peptide is from the P7-21 region of HIV-1, said P7-P1 region being defined by the sequence RQANFLGKIWPSSKGRPGNF, the peptide representing epitopes of HLA Class I A*7401 and comprising a sequence selected from the group consisting of (i) SNKGRPGNF (SEQ ID No. 6), (ii) LGKIWPSSK (SEQ ID No. 8), (iii) GKIWPSSKG (SEQ ID No. 9), (iv) SSKGRPGNF (SEQ ID No. 7), (v) GRIWPSNKG (SEQ ID No. 10), (vi) LGKIWSSNK (SEQ ID No. 11), (vii) RQANFLGKI (SEQ ID No. 12) and (viii) RQANFLGRI (SEQ ID No. 3). We contemplate the peptides (i) SNKGRPGNF (SEQ ID No. 6), (ii) LGKIWPSSK (SEQ ID No. 8), (iii) GKIWPSSKG (SEQ ID No. 9) as high-affinity epitopes for A*7401, whereas the peptides (iv) SSKGRPGNF (SEQ ID No. 7), (v) GRIWPSNKG (SEQ ID No. 10), (vi) LGKIWSSNK (SEQ ID No. 11), (vii) RQANFLGKI (SEQ ID No. 12) and (viii) RQANFLGRI (SEQ ID No. 3) could be low affinity epitopes for A*7401.

In another aspect, the invention provides an isolated peptide which is an HLA Class I-restricted peptide ligand. In particular, certain peptides represent T cell epitopes from the gag region of HIV-1, said gag region being defined by the sequence in FIG. 3. The peptide representing epitopes of various class I alleles listed in Table 4.

Another aspect of the present invention is a method of preparing a composition comprising making either an amino acid sequence designed according to the above methods or a vector construct capable of expressing that sequence in a patient, which is able to inducing a specific T-cell response in a patient infected with a micro-organism or at risk of infection with that microorganism, and then combining the therapeutic with a pharmaceutically acceptable excipient.

The invention also describes compositions for inducing a T-cell response to HIV, the compositions comprising either the peptide identified by the above methods or a vector construct capable of expressing the peptide sequence in a subject. Such peptides and constructs should be useful for inducing a specific T-cell response in an HIV-infected subject or in a subject at risk of HIV infection. The composition may include a pharmaceutically acceptable excipient and/or a carrier such as physiologic saline, and/or an adjuvant.

The invention also describes methods for inducing a T cell response against an antigen by administering to a subject either the peptide identified by the above methods or a vector construct capable of expressing the peptide sequence in the subject to induce a specific T-cell response. The cellular response may be a CD8+ T cell response, a CD4+ T cell, or both a CD8+ T cell and a CD4+ T cell response.

The peptide ligands representing the T cell epitopes as identified herein may be employed for the various diagnostic, medical and research tool applications described. However, they need not be identical to the specific epitope sequences. In some instances it may be desirable to combine two or more amino acid sequences which contribute to stimulating specific T-cell responses in one or more patients or histocompatibility types. The amino acid sequences in the composition can be identical or different, and together they should provide equivalent or greater biological activity than the parent amino acid sequences. For example, using the methods described herein, two or more amino acid sequences may define different or overlapping T-cell epitopes from a particular region, which amino acid sequences can be combined in a “cocktail” to provide enhanced immunogenicity of T-cell responses, and amino acid sequences can be combined with amino acid sequences having different MHC restriction elements. This composition can be used to effectively broaden the immunological coverage provided by therapeutic, vaccine or diagnostic methods and compositions of the invention among a diverse population. In some embodiments the T-cell inducing amino acid sequences of the invention linked by a spacer molecule, or the T-cell amino acid sequences may be linked without a spacer.

The peptide ligands of the invention can be combined via linkage to form polymers (multimers), or can be formulated in a composition without linkage, as an admixture. Where the same amino acid sequence is linked to itself, thereby forming a homopolymer, a plurality of repeating epitopic units are presented. When the amino acid sequences differ, e.g., a cocktail representing different antigen strains or subtypes, different epitopes within a subtype, different histocompatibility restriction specificities, or amino acid sequences which contain epitopes, heteropolymers with repeating units are provided. In addition to covalent linkages, noncovalent linkages capable of forming intermolecular and intrastructural bonds are also contemplated.

For pharmaceutical compositions, the peptide ligands of the invention as described above can be administered to a mammal already suffering from or susceptible to the disease being treated. Those in the incubation phase or the acute phase of disease such as a viral infection, can be treated with the immunogenic amino acid sequences separately or in conjunction with other treatments, as appropriate. In therapeutic applications, compositions are administered to a patient in an amount sufficient to elicit an effective T-cell response to the disease and to at least partially arrest its symptoms and/or complications. An amount adequate to accomplish this is defined as “therapeutically effective dose.” Amounts effective for this use will depend on, e.g., the amino acid sequence composition, the manner of administration, the stage and severity of the disease being treated, the weight and general state of health of the patient, and the judgment of the prescribing physician, but generally range for the initial immunization (that is for therapeutic or prophylactic administration) from about 1.0 μg to about 50 mg, preferably 1 μg to 500 μg, most preferably 1 μg to 250 μg followed by boosting dosages of from about 1.0 μg to 50 mg, preferably 1 μg to 500 μg, and more preferably 1 μg to about 250 μg of amino acid sequence pursuant to a boosting regimen over weeks to months depending upon the patient's response and condition by measuring specific T-cell activity in the patient's blood. Single or multiple administrations of the compositions can be carried out with dose levels and pattern being selected by the treating physician.

For therapeutic use, administration should begin at the first sign of disease (e.g., HIV infection), to be followed by boosting doses until at least symptoms are substantially abated and for a period thereafter. In cases of established or chronic disease, such as chronic HIV infection, loading doses followed by boosting doses may be required. The elicitation of an effective T-cell response during early treatment of an acute disease stage will minimize the possibility of subsequent development of chronic disease such HIV carrier stage.

The peptide ligands of the invention are confirmed by functional assays based on HLA binding. However, in order to be useful as a therapeutic, the peptides are tested in disease models. The ability of vaccines derived from peptide ligands to induce an immune response in vivo can be evaluated in models such as transgenic mice expressing human MHC class I or class II molecules such that the animals can develop physiologically relevant HLA-restricted T cell responses. Transgenic mouse strains that express either the entire HLAA* 0201 or DRB*0101 molecule have been developed. HLA-A2 transgenic mice have been used to assess the immunogenicity of peptides that bind to HLA-A2.

For HIV infection, a number of different animal model systems are known. Non-human primates such as chimpanzees and pig-tailed macaques can be infected by HIV-1. Although CD4+ cells are not depleted in these systems, the animals are detectably infected by the virus and are useful in determining the efficacy of HIV therapeutics. The animals can be inoculated with a therapeutic derived from the peptide ligands of the present invention and later challenged with a dose of infectious virus. Efficacy of the therapeutic may be determined by methods known by those of skill in the art. Generally, a variety of parameters associated with HIV infection may be tested and a comparison may be made between vaccinated and non-vaccinated animals. Such parameters include viremia, detection of integrated HIV in blood cells, loss of CD4+ cells, production of HIV particles by PBMC, etc. The therapeutic will be considered effective if there is a significant reduction of signs of HIV infection in the vaccinated versus the non-vaccinated groups.

In another aspect, the invention describes a method for validating a test peptide as an epitope of HLA Class I, the method comprising (a) contacting the peptide identified as described above with an HLA Class I molecule and detecting binding, thereby defining the control level of binding; and (b) contacting the test peptide with the HLA Class I molecule and detecting binding; wherein the level of binding in step (b) relative to step (a) determines whether the test peptide is an epitope of the HLA Class I molecule. The method may relate to the HLA Class I molecule B*1302, or A*7401 or class I alleles listed in Table 4 and wherein the binding in step (a) defines the positive control level of binding.

The invention encompasses a system or commercial package comprising components for validating a test peptide as an epitope of HLA Class I. Such a system or kit may include the peptide ligand described herein for use as a positive control with respect to HLA-restricted binding of a test peptide. The system or kit may also include the relevant HLA Class I molecules in a form suitable for peptide-restricted binding. One such system or kit is commercially known as the iTopia™ epitope discovery system.

ITopia is high throughput assay system for identifying peptides that bind to MHC, and determining their binding affinity and rate of dissociation, or off-rate. The major component of iTopia is the complex of MHC molecules, beta 2 microglobulin and placeholder peptides, bound to a streptavidin-coated microplate wells. So far, these MHC peptide complexes include eight specific Class I alleles (A*0101, A*0201, A*0301, A*1101, A*2402, B*0702, B*0801, B*1501}.

Applications with iTopia involves first, synthesizing a complete library of overlapping amino acid sequences spanning the entire length of the target polypeptide sequence. A binding assay is performed for each of the test peptides by introducing a buffer designed to unfold and disassociate the MHC and placeholder peptide in the microtitre well. The placeholder peptide and beta 2 microglobulin are washed away, leaving the unfolded MHC bound to the reaction well. A peptide from the synthesized library and additional beta 2 microglobulin are added to each well and incubated in a buffer designed to promote refolding of the complex.

A fluorescent-labeled antibody designed to recognize only a properly folded peptide/MHC complex is added to each well. This step provides the identification of those test peptides which bind to the MHC and warrant additional analysis to characterize their binding affinity and rate of dissociation. Peptides that do not bind to the MHC are clearly identified and eliminated from further study. Binding to each allele is quantified as a percentage relative to a positive control peptide for that allele. The peptide ligands as described herein, once they have been validated for binding with specific HLA alleles, can be used as positive control peptides against un-tested peptides.

High affinity binding and low dissociation rates are critical factors controlling immunogenicity of peptides. Characterization of peptide-MHC interactions can be achieved using the same plate based assay formats with slightly different assay conditions. Affinity and dissociation values for each peptide candidate are plotted in multi-parametric plots. This provides a characterization profile for the original protein designed to support the selection of potential epitope candidates and facilitate the decision making process for vaccine development.

An example of how the iTopia system may operate with the peptide ligands as described herein is as follows. A library of nonamer peptides, overlapping by 8 aa and spanning the length of a target HIV protein sequence, is synthesized. Peptide binding assays are performed to identify peptides capable of binding each HLA allele by incubating the peptides with HLA-coated wells at the set concentration of, say, 11 μM. Binding to each allele is reported as a percentage relative to a positive control peptide for that allele, e.g. the peptide RQANFLGKI. An arbitrary cutoff of 30% of the control may be applied as a positive cutoff for binders.

In another aspect, the invention provides an isolated complex comprising the peptide defined as above, bound to a HLA Class I molecule. In specific embodiments, the complex is a tetramer of HLA Class I (e.g. B*1302, A*7401, class I alleles listed in Table 4) bound to the peptide. In the tetrameric complex, the HLA Class I molecules may be conjugated to avidin to form the tetramer. The complex may further comprise a detectable label.

Soluble MHC multimers possess high avidity for T-cells since they provide multi-point binding of T cell receptors with their MHC-peptide ligands. Multimeric forms, e.g. tetramers, of MHC-peptide complexes can be used for direct phenotypic characterization of T cell responses. Recombinant soluble and secreted MHC class I and class II complexes including single chain MHC are known in the art.

In one aspect of the invention, the peptide ligands identified herein are incorporated as part of epitope-specific MHC tetramers which can bind directly to T cells that recognize the MHC-peptide complex. These reagents consist of four MHC class I molecules complexed with beta-2 microglobulin and the peptide ligand. Inclusion of a fluorochrome permits analysis by flow cytometry; e.g. the four MHC molecules may be linked together by a streptavidin molecule which may be fluorescently labeled. HLA class I tetramers could bind with sufficient avidity to HIV-specific CD8+ T cells to allow their detection by flow cytometry (77). MHC tetramers bind specifically to the T cell receptor of all T cells that recognize that particular MHC/peptide complex. They are a useful reagent for measuring antigen-specific T cells precisely, easily, and rapidly. MHC tetramers have been used extensively to visualize antigen-specific T-cell immunity in humans and in animal model systems.

MHC tetramers are specifically useful for direct ex vivo analysis of the frequency and phenotypes of epitope-specific T cells by flow cytometry. Tetramers can be used to confirm experimentally the epitope-specific T cell responses in vivo by: (1) direct quantitation of the number of epitope-specific T cells prior to and following vaccination; (2) phenotyping of responding T cells (examination for cell surface markers such as CD8, CD4, CD38, and additional activation markers); (3) monitoring of the immune response to specific epitopes following vaccination; and (4) direct evaluation of the effect of combinations of epitopes, epitope spacers or linkers and signal sequences on T cell responses. MHC tetramers provide a means of directly measuring and timing immune responses to epitope-driven vaccines.

The present invention is not to be limited in scope by the specific embodiments described herein, which are intended for the purpose of exemplification only. Functionally equivalent products, compositions and methods are clearly within the scope of the invention as described herein.

### EXAMPLE

### Materials and Methods

Study cohort: The patients were untreated HIV-1 positive adult women, at various stages of disease progression, enrolled in the Pumwani Sex Worker cohort in Nairobi, Kenya (40). This study has been approved by the Ethics Committee of the University of Manitoba and the Ethics and Research Committee of Kenyatta National Hospital. Informed consent was obtained from all women enrolled in the study.

HLA sequencing and typing: Genomic DNA was isolated from 468 HIV-1 positive women enrolled in the Pumwani sex worker cohort. HLA class I typing was conducted by amplifying HLA-A, -B and -C genes with gene specific primers. The amplified PCR products were purified and sequenced using the ABI 3100 Genetic Analyzer. The class I genes were typed using the CodonExpress software package that was developed based on taxonomy-based sequence analysis (41-43).

Gag PCR and sequencing: Proviral DNA was isolated from HIV-1 positive women. Nested PCR amplification was used to amplify gag genes. PCR amplification was confirmed via 1% agarose gel electrophoresis. The PCR products were purified using the MultiscreenHTS PCR Plate (Millipore Corp.). BigDye Terminator v3.1 was used to sequence gag genes with specific primers. The sequencing products were purified by ethanol sodium acetate precipitation. The purified sequencing products were analyzed with ABI 3100 Genetic Analyzer (Applied Biosystems). The nucleotide sequences were assembled and edited with Sequencher 4.5 (Genecodes Corp.). Samples with unsuccessful sequencing results due to heterogeneous quasispecies sequence were gel purified and cloned using TOPO TA Cloning Kit (Invitrogen). Multiple clones were sequenced as described above.

Phylogenetic Analysis Phylogenetic analysis with MEGA (Molecular Evolutionary Genetics Analysis) v3.1 was used to classify viral subtypes. All of the sequences were aligned using Clustal W (44), along with reference sequences obtained from the Los Alamos HIV database (45). Phylogenetic trees were constructed using Neighbor-joining algorithms with bootstrap test of 1000 replicates. RIP (Intersubtype Recombination Analysis) v2.0 (45) was used to identify inter-subtype recombinations. Shannons' entropy was used to score the sequence variability in our gag protein alignments using the procedure described by Korber et al (46). The score considers both the number of amino acid variants and their frequencies for each position, providing a quantitative measure for comparisons of gag sequences

Positive selection analysis: We used QUASI, a selection mapping algorithm (39), to identify the positively selected amino acids of viral proteins. This selection-mapping program identifies replacement mutations that are over-abundant compared to silent mutations at each codon, recognizing them as positively selected (39).

Proteasomal cleavage prediction: The NetChop C-term 3.0 calculated cleavage values for gag residues and identified potential proteasomal cleavage sites (47,48). A low cleavage value indicates a low probability of proteasomal cleavage; a high value suggests a high probability of cleavage.

TAP affinity prediction: Predicting the TAP affinity, and therefore the TAP transport efficiency, was performed using the consensus scoring methods described by Peters et al. (49). The TAP affinity score is the sum of the matrix elements of the C-terminus and three N-terminal residues, for any arbitrary length represented by log (IC50) values. This equation optimally applies to nonamers, however it was also highly correlated to peptides with 10 to 18 amino acids. A low TAP score corresponds to a peptide well suited for TAP binding, and a high TAP score corresponds to low TAP affinity.

Statistical analysis: All statistical analyses were done using SPSS 11.0. Pearson's chi-square test was used to correlate positively selected amino acids with HLA class I alleles. Comparison of mean CD4 counts between positively selected amino acid and the consensus was conducted using independent-samples t test.

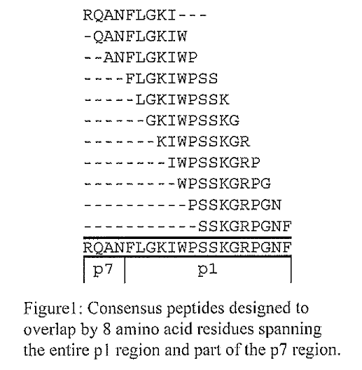

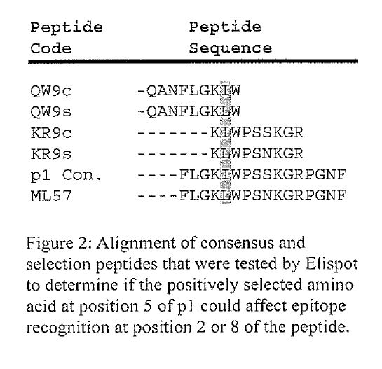

Epitope Confirmation by ELISPOT: The potential epitopes identified by correlation analysis were confirmed by ELISPOT using patient PBMCs. We select p7-p1 region containing several positive selected amino acids that are highly correlated with three HLA alleles, B*1302 (p=2.98E-11), A*7401 (p=2.46e-4), and A*30 (p=4.65e-3) for initial epitope confirmation. Overlapping peptides were designed in sequences of 9 amino acid residues overlapping by 8, to span part of the p7 region and the entire p1 region, a total of 16 amino acid residues (FIG. 1). The overlapping peptides comprise the consensus sequence as well as mutations at various positively selected amino acid residues and each positively selected amino acid can be found in the anchor positions 2 and 8 of the 9mer peptide. An example is shown in FIG. 2 for a mutation in position 5 of p1 from Isoleucine to Leucine.

The testing peptides are selected based on the p1 sequences found in the given patient in addition to the consensus peptides in all assays. For each selected peptide, 105 PBMCs (peripheral blood mononuclear cells) were used and tested in duplicate.

The peptide stock (4000 μg/ml in 100% DMSO) is diluted to a working solution at 20 μg/ml with RPMI and frozen down in aliquots. The ELISPOT assay was carried out following standard procedure. The air-dried plates were read using an automated ELISPOT reader. Results were considered positive when the SFU/million was greater than or equal to 50. In some patient samples an interferon gamma co-stimulatory molecule was added to the cells to facilitate production once activated by a peptide.

### Results

### 1. Identify Potential Epitopes Through Correlation Analysis

### Identification of Positively Selected Amino Acid Via QUASI Analysis.

Positively selected amino acids identified by QUASI were used to characterize the selection landscape of p17, p24, p7, p1, and p6 (FIG. 3). Because of the significant genetic distance and complications of interclade variability between clades A1 and D, we conducted QUASI analysis separately for each clade. Clade A1 and D subtypes differed in the number of positively selected sites, amino acid variants and frequencies of variants. We generated a positive selection map across gag (FIG. 3). These mapped positively selected amino acids were then analysed for selection pressures that may drive viral evolution at the various avenues of escape.

### Determining HLA Escape Mutations.

Mutations in the anchor positions may lead to a reduction in HLA binding, and abolish peptide presentation (50). We used Pearson Chi-square to correlate the positively selected amino acids with the patients' HLA alleles (Tables 1 and 2). This analysis permits identification of potential epitopes for rare HLA alleles and undocumented epitopes. Overall, 39 positive and 8 negative HLA correlations were observed in p17 clade A1 (Table 1), while 11 positive and 2 negative HLA correlation were found in p17 clade D (Table 2). 32 of these HLA correlation have not been classified by previous studies. The results are summarized in Table 3. K28Q, in p17, is located within the beta sheet basic domain between helices 1 and 2. It is significantly associated with HLA-A*0301 genotype (P=1.65E-16, see Table 1) which is known to bind the epitopes 18KIRLRPGGK26 (KK9) (51) and 20RLRPGGKKK28 (RK9) (9,36). Since K28Q is located in the C-terminal of the epitope RK9. It is possible that this mutation may also result in a reduction in HLA-A*0301 recognition.

The HLA-A*0201-restricted epitope SL9 had several positive selection sites including F79Y, T1A, V82I, A83V, and L85I. Significant associations were found for HLA-A*02 to sites F79Y (P=5.56E-04), LS5I (P=4.61E-03), and an upstream residue L75I (P=1.68E-05). Site F79Y had significant positive associations with HLA-A*0202 (P=2.26E-05) and HLA-B*5703 (P=5.67E-03). This site also had significant negative associations with other alleles (see Table 1). HLA-A*01 (71GSEELRSLY79), HLA-A*0201 (SL9), and HLA-A*3002 (76RSLYNTVATLY86) epitopes were previously identified in this region (45). The negative associations suggest that the consensus phenylalanine is an escape mutation that has become fixed in the population (29).

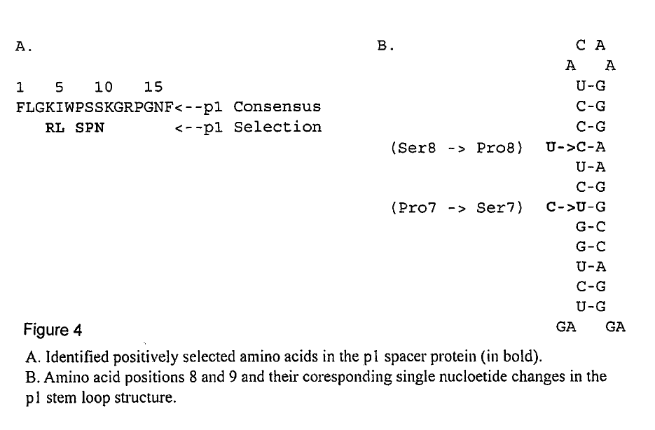

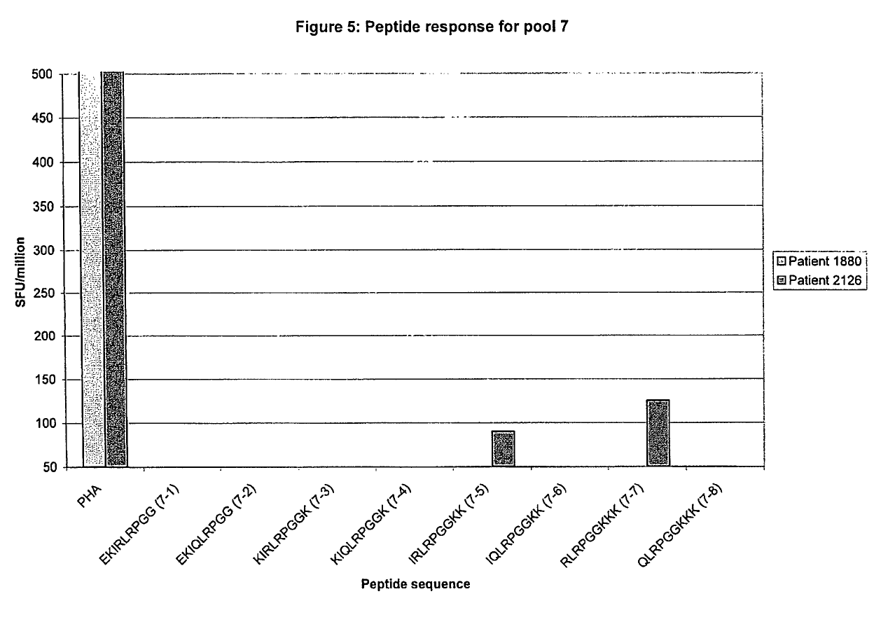

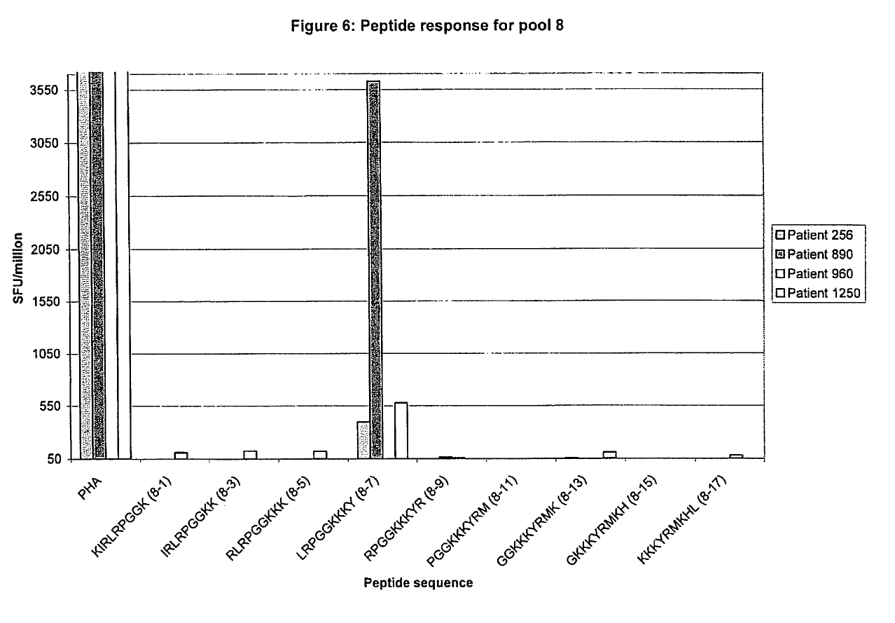

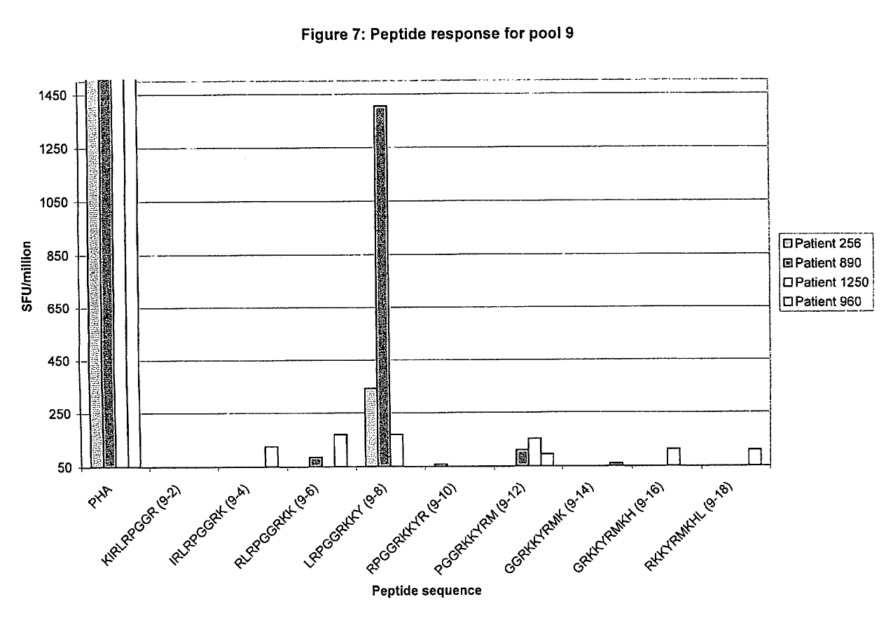

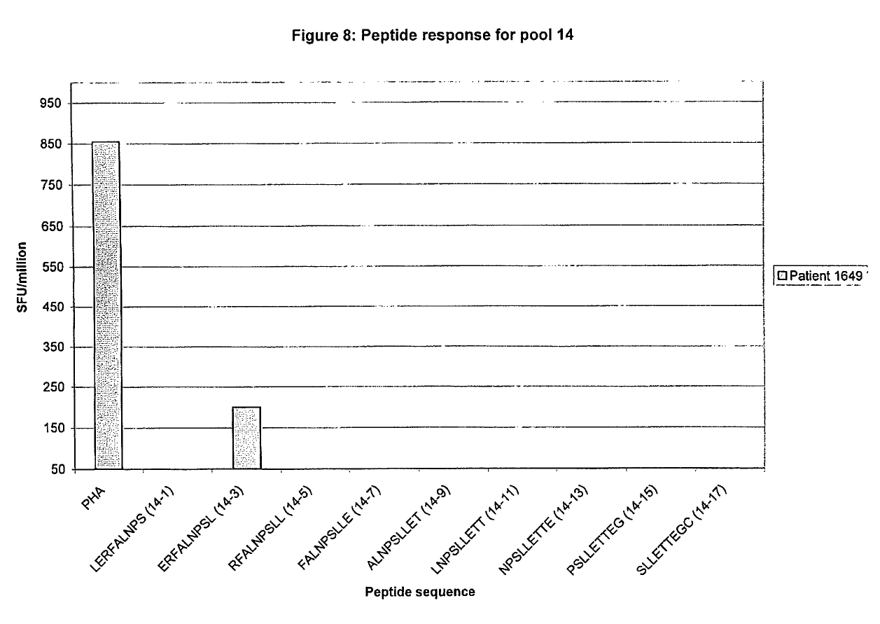

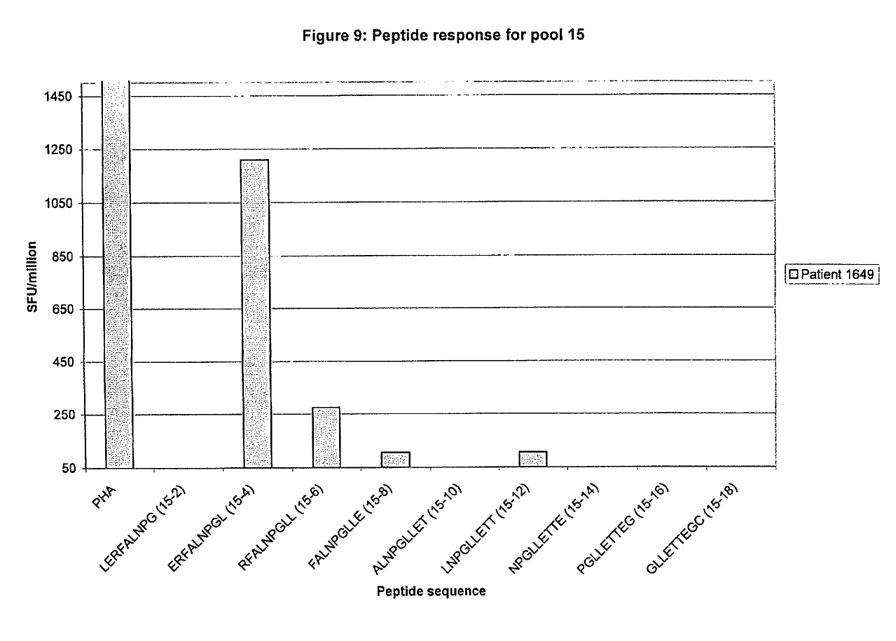

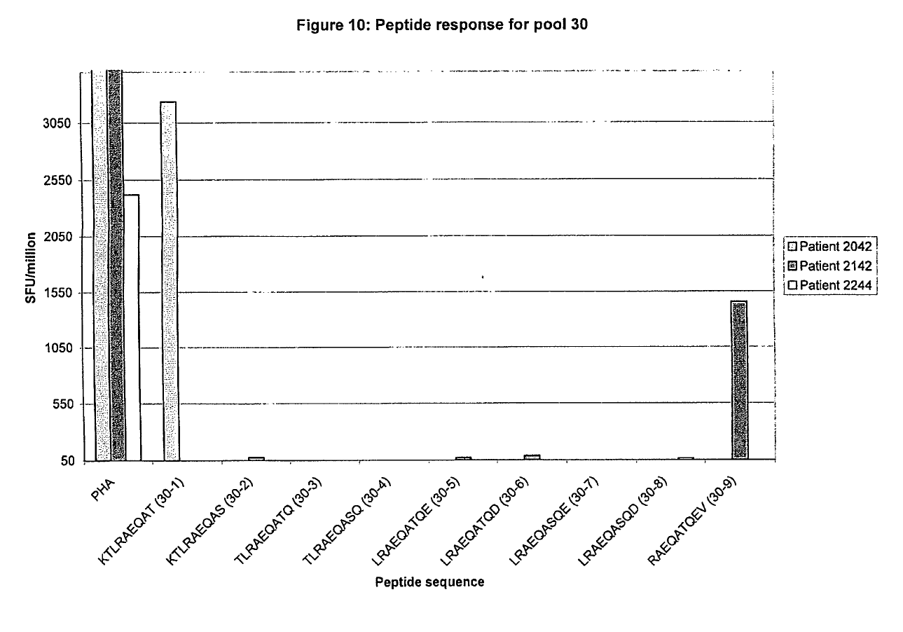

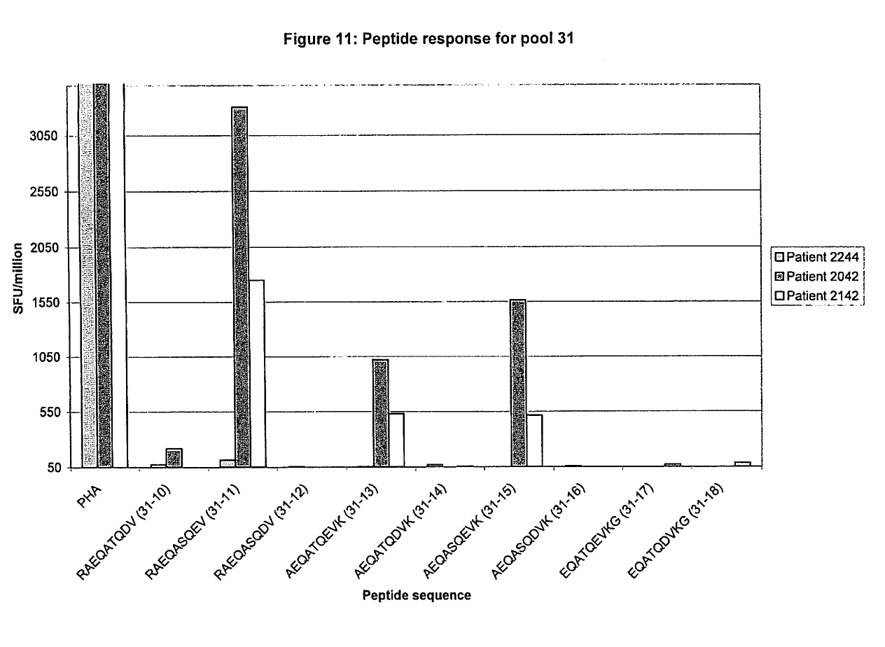

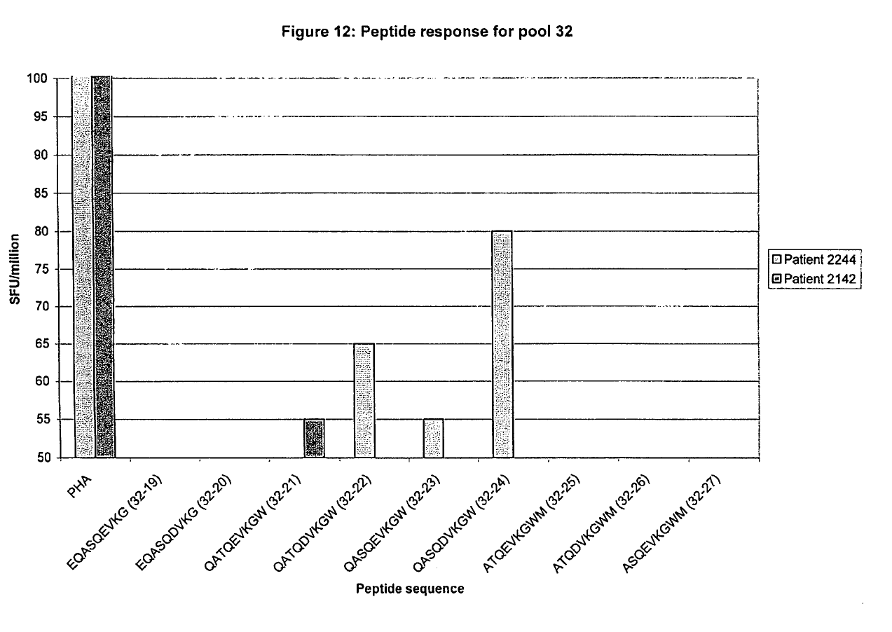

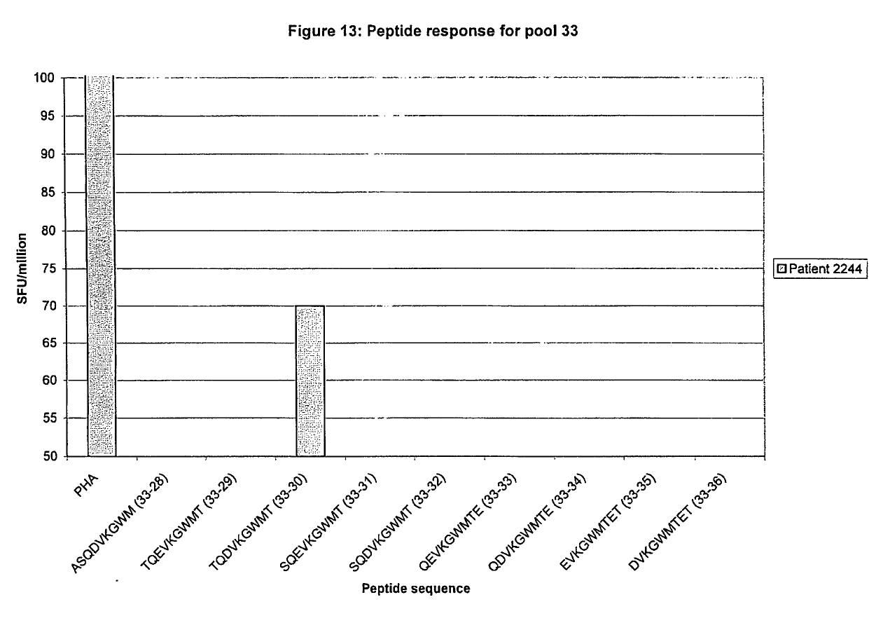

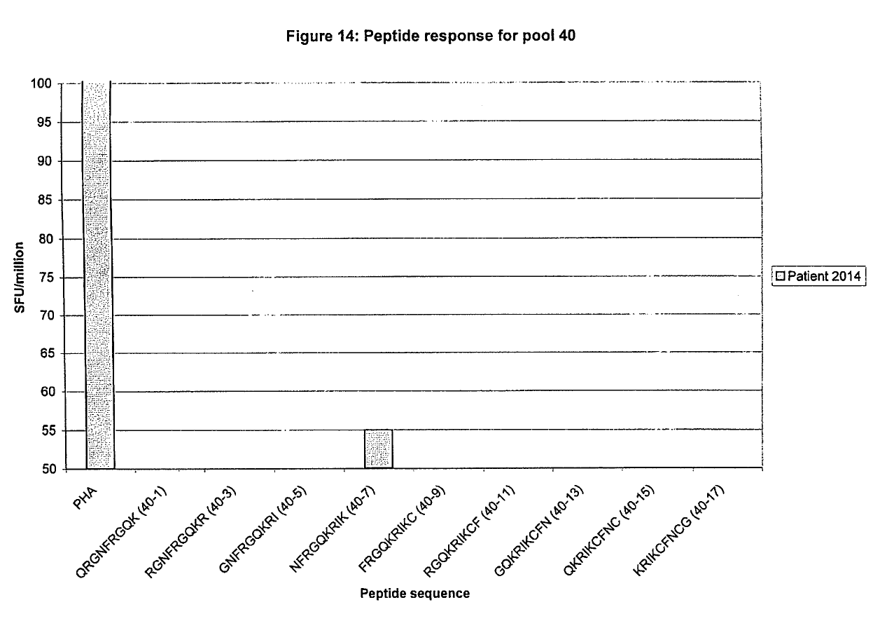

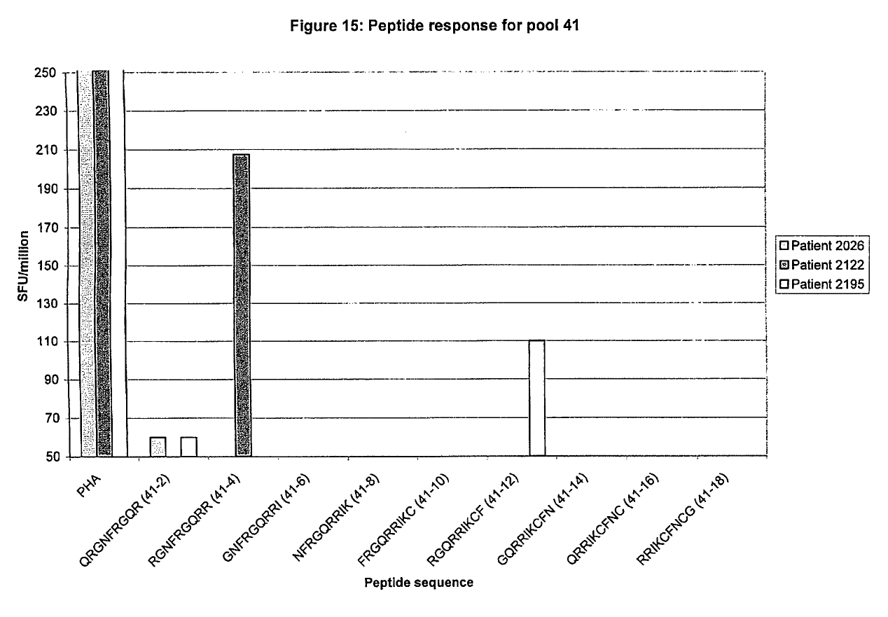

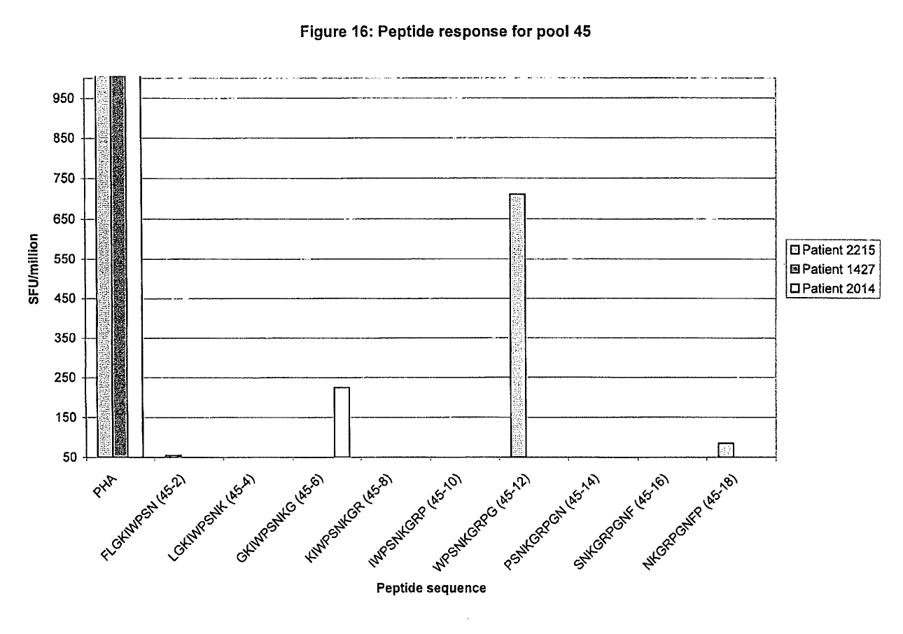

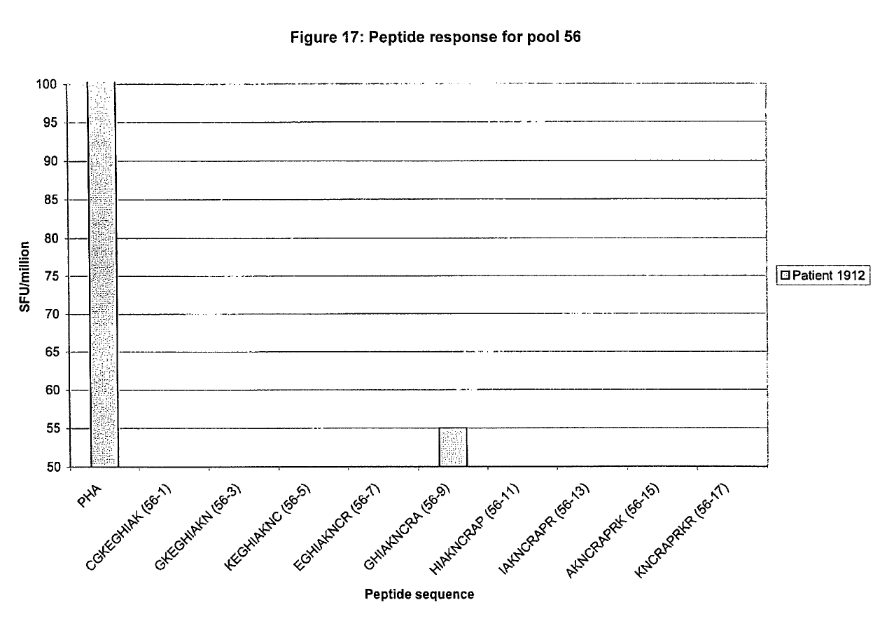

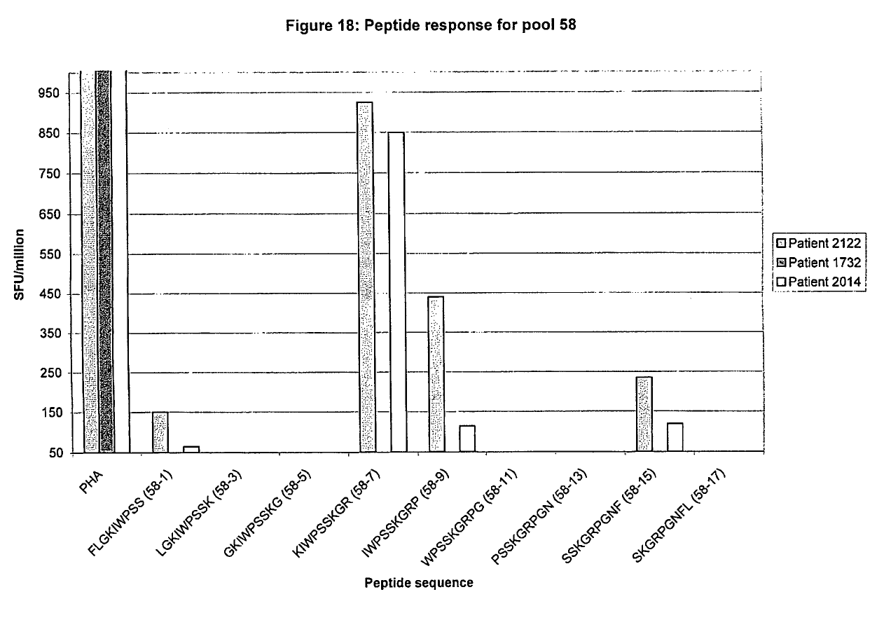

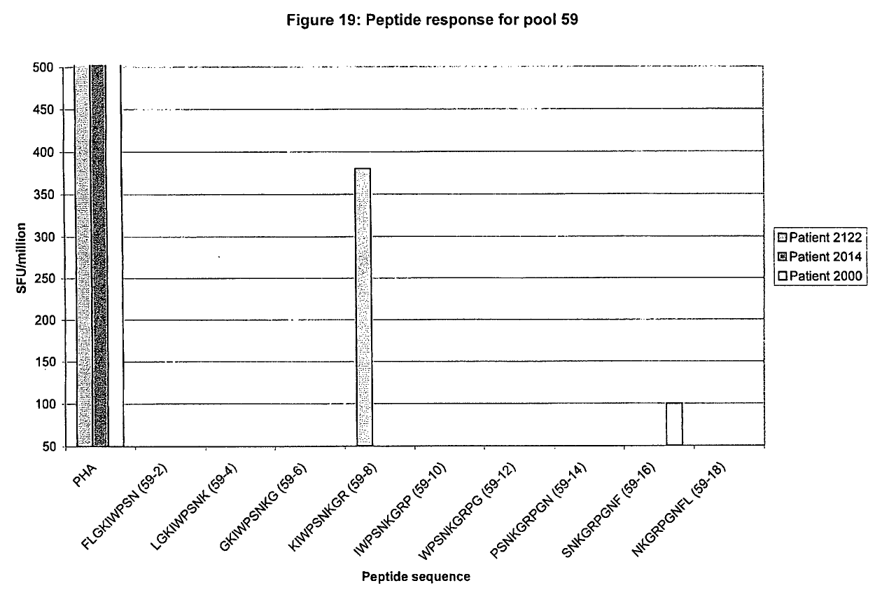

According to Table 1, positively selected mutation L85I of SL9 is associated with HLA-A*02 (P=4.61E-03), A*0240 (P=1.25E-04) and HLA-A*0201 (P=1.83E-03), and resides at the C-terminal anchor of their epitope (FIGS. 3 and 4). This suggests that the L85I mutation is an escape variant from HLA-A*02 recognition. Also, positively selected mutations flanking SL9, such as L75I and I92M (positively correlated to HLA-A*02, P=1.68E-05 and 3.12 E-03, respectively), are possible escape mutations that may prevent both peptide processing and recognition of SL9 epitope.

We next summarized the effect of HLA alleles associated with long-term non-progression on HIV-1 evolution by analysing and classifying positive selection in gag proteins of the Kenyan cohort. HLA alleles such as B*5701 and B*5703 have been strongly associated with slow progression to disease and consequently exerts a strong selective pressure on the virus (52). Five immunodominant epitopes restricted by HLA-B*57 have been located in the p24 region: 147ISPRTLNAW155 (IS9), 162KAFSPEVIPMF172 (KAF11), 162KAFSPEVI169 (KAF8), 240TSTLQEQIAW249 (TW10), and 308QASQEVKNW316 (QW9) (29,53-55). The positively selected mutation in clade A1, A163G of the p2 anchor position of the KAF11 and KAF8 epitopes is highly correlated with patients expressing HLA-B*5703 (P=1.96E-10). This mutation is also associated with an increase in CD4 counts from 337.52 cells/ml to 425.56 cells/ml (P=0.038). It has been shown that HLA-B*5703 can still present A163G epitope variant efficiently (15). This could be a basis for the association of HLA-B*5703 with slower disease progression.

Using this method we can also identify the viral sequences correlated with HLA alleles associated with rapid disease progression, such as HLA-B*5802 (30). HLA-B*5802 in the Kenyan cohort is highly correlated with the positively selected mutation at residue 190 from Ile to Val (P=1.36E-04) in sub-type A1 infected individuals. This mutation correlates to a decrease in mean CD4 count from 364.04 to 271.73 CD4 cells/ml (P=0.019), suggesting that viral strains with V190 are more fit than strains with I190. This mutation is also negatively correlated to HLA-Cw*07 (P=0.002), implying selective pressure towards consensus in its presence. Similarly, in p17 A1 subtype, the mutation T81A was associated with HLA-B*5802 (P=8.31E-03), and also negatively associated with HLA-B*5801 (P=2.27E-04) an allele associated with long-term non-progression 30. This mutation is also correlated to a lower mean CD4 count (256.61 cells/ml, compared to 354.69 cells/ml of those with T, P=0.0073).

Although there are many sites within the p7-p1-p6 region of gag that have been identified as being subject to selection pressure, and many of these sites correlate with specific HLA alleles, few of them are within the anchoring residues of the presently documented HLA epitopes. In the D clade p7 sequences, the positively selected mutation R406K resides in the anchor position of the HLA-B*14 epitope 405CRAPRKKGC413 (CC9), however the SYFPEITHI database (56) identifies Arg, Lys and His as dominant amino acids at this position, and so R406K would be unlikely to affect recognition by HLA-B*14. The Kenyan viral population contains the sequence 40CRAPRKRGC413 at this location with positive selection at residue 411 toward Lys. This positive selection appears to be beneficial to the virus as is discussed later (see next section) and demonstrates CTL restricted viral evolution. Positive selection at residues 436 (K436R) and 437 (I437L) of p1 correlates strongly with the presence of the HLA-B62 supertype (P=6.80E-05 and 3.0E-04 respectively) indicating a possibility that an epitope resides within the region.

Viral mutations that spare the anchoring residues of an epitope should not affect HLA binding, however viral evasion might still occur due to impaired recognition of the HLA-peptide complex by the TCR (54).

In the Kenyan cohort The V82I mutation within the SL9 epitope restricted by HLA-A*0201 correlated to a decrease in CD4 counts from 351.13 cells/ml to 277.48 cells/ml although this result was not significant (P 0.142). Similarly, it was reported in a longitudinal study that V82I escape mutations arose within two weeks of selection pressure from a gag specific CTL clone (36).

In the investigation of non-anchor mutations within known p24 epitopes, we have observed in the clade A1 sequences a positive selection in residue 218 that is significantly correlated with a change in CD4 counts (Table 1). This mutation is located in the P3 position (position 3 of epitope) of the ELA-B*3501-restricted epitope, in which a change from Val to Ala significantly associates with a decrease of mean CD4 count from 360.50 to 222.46 CD4 cells/ml (P=2.58E-04). Another indication of a loss of CTL recognition is also seen in the clade D sequences. The mutation occurs in amino acid 255, which is located in the P6 position within HLA-A*0201-restricted epitope. This Pro to Ala mutation also correlates with a decrease in mean CD4 count from 334.03 to 116.33 CD4 cells/ml (P=0.002).

Several positively selected sites within p7 gag were also observed to associate with lower CD4 counts. Amongst the D clade viruses, the mutation R411K occurs in the seventh residue of the HLA-B*14 restricted epitope 405CRAPRKKGC413 and is associated with a lower mean CD4 count. As noted earlier, our consensus at this site is 405CRAPRKRGC413 with positive selection towards Lys at the seventh position. Those with the consensus Arg have an average of 400.65 CD4 cells/ml, while those containing the R411K mutation (46% D clade virus) have an average of 275.97 CD4 cells/ml (P 0.024). This observation suggests that R411K decreases recognition by the TCR.

The p7 mutation K387R correlates very strongly with HLA-A*7401/02 among those with A1 clade virus (P=3.50E-05). The nearby mutation R384K also correlates well with A*7401/02 (P=0.009), but appears to occur in tandem with the K387R mutation (P=5.10E-11). R384K is associated with a lower mean CD4 count (315.96 cells/ml of those with Lys compared to 386.17 cells/ml of those with consensus Arg, P=0.016) implying that this mutation results in faster disease progression.

Our analysis identified many potential epitopes for various HLA alleles that have not been documented before. We have assigned peptide sequences for these potential epitopes and summarized them in Table 4.

### Determining Proteasomal Escape Mutations.

NetChop 3.0 at a threshold value of 0.5 was utilized to assess selections that may abrogate viral peptide processing for HLA class I presentation. However, this prediction algorithm only determines C-terminal cleavage sites because the determination of N-terminal cleavage sites is more complicated (48). Positively selected mutations that occur on the C-terminal cleavage sites may abolish proteasomal processing, and any such mutations that flank the C-terminal cleavage site and occur within 14 amino acid residues may also affect cleavage. Therefore, we analysed positively selected mutations that occur at the C-terminus of the epitope and residues within, or flanking the epitope. The positively selected mutations affecting the proteasomal cleavage are marked in FIG. 3.

In p17 of subtype A1, the HLA-A3-restricted RK9 epitope contained a positive selection at the C-terminal anchor residue K28Q (FIG. 3). Conserved Lys to Gln mutations at this site abolish proteasomal cleavage as indicated by the dramatically reduced NetChop score (from 0.625 to 0.093). The K28Q mutation at the C-terminal proteasome cleavage site is not affected by other mutations within fourteen-residue sequence range. This suggests that the K28Q mutation is a proteasome escape variant. Patients carrying the K28Q mutation tend to have a lower CD4 count, however the difference was not significant (data not shown). Our observation is consistent with a previous study that suggests the K28Q mutation might impair the HLA-A3-restricted epitope processing and reduce binding (5).

The extended peptide generated from proteasomal cleavage requires the N-terminal trimming in the ER, an essential step in peptide processing (57,58). For instance in p24, the positively selected amino acid of A146P flanking the immunodominant HLA-B*57-restricted epitope IW9 (p24: 147-155) was found previously to prevent N-terminal trimming by the ER aminopeptidase I (59). In the Kenyan HIV population, the A146P mutation in clade D is associated with a lower CD4 count (from 344.68 to 283.52 cells/ml) although it is not statistically significant (P<0.25). Another positively selected amino acid, I147L, located within the same epitope is significantly associated with a decrease in CD4 count (from 356.36 to 283.52 cells/ml, P=0.014). Goulder et al (60) found that A146P and I147L occurred at the same time in HLA-B*57 progressors. We also observed that A146P and I147L simultaneously occurred in 11% of the Kenyan patients and was associated with HLA-B*57 (P=2.80E-10) and a significant decrease in CD4 count from 372.86 to 231.00 cells/ml (P=0.009).

Analyses of Kenyan p7 samples reveal no positively selected mutations that can alter proteasomal cleavage sites within the two C-terminal residues containing the conserved zinc fingers and the basic linker peptide (61,62). Kenyan A1 clade viruses only have changes to the predicted cleavage sites within the first 12 residues of the protein. The R380K mutation is predicted to result in a removal of one of these sites at residue 387 with a concomitant formation of a new site at residue 380. Likewise, amongst the D clade viruses the positively selected mutations from residues 386 through 389 results in a variety of proteasomal cleavage sites either being created or destroyed, however as was the case with A1 clade viruses, they all occur within the first 12 residues. There are no known epitopes that have been identified in the region of first 12 residues of p7. The I401T and R418K mutations in the D clade viruses result in the creation of cleavage sites at residues 397 and 418 respectively, however these new sites occur outside of documented epitopes and are not likely to influence the processing of the known epitopes.

### Determining TAP Escape Mutations.

A TAP affinity algorithm was used to determine TAP-peptide binding log IC50 scores (49) (Table 5) and predict TAP escape mutations. The higher log IC50 value indicates a lower TAP-peptide binding affinity. Positive selection was observed in the A3 restricted epitope RK9 at sites 1 and 9 (R20Q and K28Q respectively). The change from RK9 to RQ9 resulted in an increase of log IC50 value from −2.12 nM to −1.55 nM (a difference of 0.57 nM) while RK9 to QK9 resulted in a greater increase of log IC50 value from −2.12 nM to −0.31 nM (a difference of 1.81 nM). The patients who have K28Q mutation (RQ9 fragment) showed lower mean CD4 counts (Table 5) suggesting RQ9 is a TAP transport escape variant. Whereas, the patients with QK9 mutation tend to have a higher mean CD4 count suggesting a substantial fitness cost to the virus.

We have also observed a positive selection in p24 that is indicative of a TAP escape mutation. In clade D, a change in amino acid residue 252 from Ser to Asn within the HLA-A*0201-restricted epitope, increases the log IC50 value from −0.61 to 0.29 nM, corresponding to a decrease in binding affinity. This change in TAP binding affinity is correlated with a significant decrease in mean CD4 count from 348.32 to 271.45 cells/ml (P=0.050).

The p7 of the D clade contains a positively selected mutation that seems to affect TAP binding. The HIV database lists the region 401LARNCRAPRK410 (LARK10) as an epitope to HLA-A*03. In the Kenyan cohort the D clade consensus sequence at this site is 401IAKNCRAPRK410, (IAKK10). The mutation, K403R, correlates to HLA-A*030101 (P=0.001) indicating that this sequence is also potentially under selection pressure by HLA-A*03. The TAP binding score (logIC50) for the LARK10 epitope is −1.62 nM, a score that indicates excellent affinity for TAP. IAKK10 increased the TAP score by almost two log units to 0.09 indicating a substantial decrease in TAP affinity. This suggests that adaptation has occurred at this site for clade D virus in this cohort.

### 2. Confirmation of Potential Epitopes by ELISPOT Assay

We used ELISPOT assays to ensure that the patients with the HLA alleles that significantly associated with the positive selected amino acids can actually recognize the peptides identified through correlation analysis. For p1 spacer protein, QUASI analysis identified positively selected amino acids at positions 4, 5, 7, 8 and 9 of the p1 spacer protein (FIG. 4). The positively selected Arginine and Leucine at positions 4 and 5 seem to be conserved substitutions while the positively selected Serine (7), Proline (8) and Asparagine (9) are not. The region was reported to contain an A*0201 epitope (63) but no epitopes are known for other alleles. Two of five identified positive selected amino acids in p1 spacer protein (pa4 and pa5) are significantly associated with B*1302, while another two positive selected amino acids (pa7 and pa9) are significantly associated with A*30 and A*7401 respectively.

Correlation Analysis of Positively Selected Amino Acids in p1 with Patient HLA Alleles Identified Potential Epitopes for HLA-B*1302

Chi-square analysis was carried out to identify the associations between the positively selected amino acids of the p1 protein with patient HLA class I alleles. Several alleles are significantly associated with the positively selected amino acids in p1. A strong correlation was found between HLA-B*1302 and the amino acid at position 4 in p1 (p-value=1.96e-009) (Table 6). A similar association was also observed between HLA-B*1302 and the positively selected amino acid at position 5 (p-value=2.98e-011) as shown in Table 7. The strong correlations of positively selected amino acids at positions 4 and 5 of the p1 spacer protein with B*1302 suggest that the p1 protein contains a B*1302 epitope. The possible epitope could have the positively selected amino acids at anchor position 2 or 8. Mutation at these positions could either weaken or abort the binding of the peptide by B*1302.

### Potential Epitopes Identified by Correlation Analysis are Confirmed by ELISPOT for B*1302

ELISPOT results confirmed the potential epitopes identified through correlation analysis. A summary of ELISPOT responses is shown in Table 8. The detailed ELISPOT data of each patient can be found in the attached appendixes. The consensus peptides are illustrated in bold and the number of patients that responded listed on the right. The HLA class I alleles of patients who were tested and responded to peptides RQANFLGKI (40) and RQANFLGRI (41) are listed in Table 9. Of the 8 patients tested for these two peptides, 7 of them responded (87.5%) to both peptides. The low value in the positive controls of patient ML1728 suggests that the poor condition of the cells might be the reason for the undetectable response. Therefore, all viable PBMCs from patients with B*1302 responded to the two peptides and EQANFLGKI (40) and RQANFLGRI (41) are the epitopes of B*1302. The only difference between these two peptides is in position 8 of the peptide and both “R” and “K” are positively charged and hydrophilic. The lysine to arginine is a conserved substitution and should not affect peptide binding. In this case both peptides are recognized by B*1302.

The “K” and “R” at anchor position 8 and “Q” at position 2 are critical for B*1302 recognition because there is no recognition to peptides QANFLGKIW (36) and QANFLGKLW (37) even though there is only one amino acid difference between these two peptides and B*1302 epitopes, RQANFLGKI (40) and RQANFLGRI (41).

Peptides KIWPSSKGR (14) and KLWPSNKGR (16) were tested by the ELISPOT assay in 7 patients, 6 of which (85.7%) responded to either KIWPSSKGR (14) or KLWPSNKGR (16). The differences between these two peptides are positions 2 and 6. Some patients can recognize both peptides while others can only recognize one of the two. The low value in the positive controls of patient ML1728 suggests that the poor condition of the cells might be the reason for the undetectable response. The HLA class I allele compositions of these patients are listed in Table 10. Similarly, 5 out of 7 (71.4%) patients tested for peptides SNKGRPGNF (43) and SSKGRPGNF (44) responded to either SSKGRPGNF (44) or SNKGRPGNF (43). The difference between these two peptides is serine versus asparagine, both are hydrophilic residues, however they don't appear to be interchangeable in that no patients responded to both. HLA data can be found for these patients in Table 11. The consensus peptide LGKIWPSSK (20) was tested in all 10 patients by ELISPOT and 40% of them responded. Again, the values of the positive control in patient 1728 is low, it may account for the lack of response. HLA data of the patients can be found in Tables 10-12.

In summary, ELISPOT analysis of PBMCs from patients with B*13 genotype showed that all of the B*1302 patients with viable PBMCs recognized peptides RQANFLGKI and RQANFLGRI. Peptides KIWPSSKGR or KLWPSNKGR was recognized by 85.7% of the patients with B*1302 or B*1301 allele. Peptides SNKGRPGNF or SSKGRPGNF was recognized by 71.4% of patients, and 40% of the patients recognized peptide LGKIWPSSK.

Correlation Analysis of Positively Selected Amino Acids in p1 with Patient HLA Alleles Identified Potential Epitopes for HLA-A*7401 and A*30

Correlations between HLA and positive selected amino acids in p1 identified a significant association between HLA-A*30 genotype and amino acid 7 (p-value=4.65e-3) from Proline to serine as shown in Table 13. This proline residue is important for stability of the stem loop. Similarly, a significant association (p-value=2.46e-4) was found between HLA-A*7401/02 and amino acid residue 9 from serine to asparagine as shown in Table 14.

### Potential Epitopes Identified by Correlation Analysis are Confirmed by ELISPOT for A*7401

Results of the ELISPOT assays for A*7401 patient samples confirmed potential HIV-1 epitopes that were identified by correlation analysis. A summary of ELISPOT responses is shown in Table 15. The consensus peptides are indicated in bold and the number of patients that responded listed to the right.

Peptides SNKGRPGNF (43) and SSKGRPGNF (44) were tested by the ELISPOT assay in 15 patients, 10 of which (66.7%) responded to one or both peptides. One patient can recognize both peptides while others can only recognize one of the two. The difference between these two peptides is in position 2, where the consensus ‘S’ is mutated to an “N”. More often the response was to the peptide 43 containing the positive selected amino acid rather than the consensus. Peptides SNKGRPGNF (43) can be considered a confirmed epitope of HIV-1, while peptide SSKGRPGNF (44) could be an epitope with much reduced affinity. The HLA class I allele compositions of these patients are listed in Table 16.

Similarly, 3 out of 7 (42.9%) patients tested for peptides GKIWPSSKG (10) and GRIWPSNKG (11) responded to at least one of these peptides. Interestingly the one patient that recognized both peptides, ML 672, is the same patient that recognized both peptides 43 and 44. The others were only able to recognize peptide 10, the consensus. The difference between peptides 10 and 11 occurs at positions 2 and 7 of the sequence, however the position of interest was position 2 where the ‘R’ is mutated to ‘K’. HLA class I for these patients are listed in Table 17.

Another common response is to peptides LGKIWPSSK (20) and LGKIWSSNK (21). Of the 15 patients tested, 6 (40%) of them recognized one or both of these peptides. Only ML1072 recognize both peptides. Most of the patients were only able to recognize the consensus peptide LGKIWPSSK (20). The differences between the two peptides lie in position 6 and 8 of the 9mer with position 8 being the one of interest. The class I HLA data of each patient is listed in Table 18.

Finally, 4 of 13 patients tested with peptides RQANFLGKI (40) and RQANFLGRI (41) responded to at least one of these two peptides. The one that was able to recognize both was again ML672. The difference between the peptides of interest is in position 8, where the consensus ‘K’ residue is mutated to an ‘R’ residue. See Table 19 for the patient class I HLA data.

In summary, ELISPOT analysis of PBMCs from patients with A*7401 genotype showed that more than 60% of fifteen patients with viable PBMCs recognized peptides SNKGRPGNF and 40% of them also recognize peptide LGKIWPSSK (20). In addition, 42% of the patients examined for peptide GKIWPSSKG (10) showed positive ELISPOT response. The three peptides could be the epitopes for A*7401. Whereas, positive ELISPOT response can only be detected in a small portion of the patients for peptides SSKGRPGNF (44) or GRIWPSNKG (11), LGKIWSSNK (21), RQANFLGKI (40) and RQANFLSRI (41). These peptides could be low affinity epitopes for A*7401.

### Discussion

It is well accepted that the host immune system influences the evolution of infectious pathogens that are dependent on host survival to propagate while infectious pathogens select for resistance in the host. However, the influence of the host immune system on the viral evolution can be observed in a much shorter time due to the rapid turn over of the virus in comparison with the life span of the host. It is especially true in the case of HIV-1. The imprint of the host immune system on the evolution of the HIV-1 virus can be readily observable in a very short time due to the error prone reverse transcriptase and rapid turn over time of the virus. Human Leukocyte Antigens are at the centre of the host immune system as they are responsible for recognition of the infectious pathogens and present them to CD4 and CD8 T cells. The escape mutations of HIV-1, therefore, are the results of CD4 and CD8 T cell responses initiated through HLA recognition. Conversely, peptides recognized by HLA alleles can be identified through analysis of escape mutations of HIV-1 and correlate them with host HLA alleles. In this approach it is very important to tease apart the mutation noises due to random drift from the positive/negative selected amino acids. Quasi analysis was proven to be a fast and reliable tool that can conduct such analysis with thousands of sequences efficiently. We have successfully used this approach to identify novel epitopes for B*1302, B*1301 and A*7401. These epitopes have not been identified or mis-identified by the traditional methods (25,30). With this approach we can also identify parts of the HIV-1 genome that is critical for viral function and survival.

In the example shown here, we use an integrated approach to identify suspected CTL escape mutations in HIV-1 gag. Although many studies have shown that HLA binding and CTL recognition contributes the most specificity in cellular mediated immune response, there has been increasing evidence that both proteasomal cleavage and TAP affinity may contribute as well (47,49,57,64-66). Therefore, we have integrated predictions of C-terminal proteasomal cleavage, TAP affinity, and HLA class I associations, and correlate them with CD4 count.

We have described proteasomal escape variants as those that abolish C-terminal cleavage sites upon mutations that occur in the C-terminus of the epitope, within the epitope, or flanking the epitope. However, we did not consider strong internal cleavage sites that could destroy potential epitopes as a possible escape variants, since the QUASI output is generated from a population with different HLA-restricted epitopes. It may be possible, therefore, to observe multiple cleavage sites within a single epitope. Other studies have tested the predictions of internal cleavage sites on its contribution to epitope identification when combined with predictions of MHC class I binding; however it was found that none of the internal sites could improve the ability to identify epitopes (67). Still, it is reasonable that the virus uses this mechanism to escape. For example, in a previous study the binding motifs of HLA-B*57 usually carry Phe, Trp, Ile, or Val as its C-terminal residue (68). However, NetChop 3.0 predicts an internal cleavage site on P10 Met as the C-terminus of the HLA-B*57-restricted epitope KAF11 in p24. This will impair the many interactions between the P11 Phe and the contact residues of HLA-B*5703 (55).

MHC affinity prediction servers were difficult to integrate into this study because most had limited numbers of HLA class I alleles. In addition the alleles available were mainly non-African. Many dealt exclusively with 9mers and a few with 10mers, however HLA-restricted epitopes in gag vary from 8mers to 12mers. Likewise, the TAP affinity prediction algorithm was also designed for 9mers; nevertheless we applied it to peptides with more than nine residues. As mentioned in Materials and Methods, the algorithm was applied in a previous study on peptides with lengths between 10 and 18 amino acids (49). Peters et al. found that the correlation between predicted and measured affinity value for the 10-18mers was lower than for the 9mers, but it was still significant. Another interesting study also integrated predictions of C-terminal proteasomal cleavage, TAP transport efficiency, as well as MHC class I binding affinity to identify CTL epitopes (67); however their predictions only generated 9mers, thus it was difficult to compare with their findings with ours.

Negative correlations with HLA alleles and positive selection were frequently observed in our analysis. These negative associations are thought to be a result of successful transmissions of positively selected escape mutation to the point of fixation (52). An escape variant that becomes abundant in the population will be lost as a potential target for the immune system. For example, HLA-Cw*07 is negatively correlated to 158V mutation in p24 clade A1 sequences. Ile-58 is suspected to be an escape mutation when the epitope is driven by HLA-Cw*07, a common allele in our population. This V58I mutation may have been transmitted and subsequently accumulated within the population, which led to a replacement of Val by Ile as the consensus sequence (52). Previous studies have shown that ultimately not all escape mutations accumulate in a population. Escape mutations that result in a fitness cost usually revert back to its wild type when it is transmitted to individuals lacking the particular HLA allele that exerted the selection (29,69).

The p24 capsid is a highly conserved protein and is limited to small amount of mutations. Changes in critical regions may abolish important functions and therefore be detrimental to the virus (53). We investigated the cyclophilin A binding loop in p24, in which we observed a positively selected amino acid substitution at residue 223 from Ile to Val (n=89) in clade D infected individuals. However, this amino acid change does not correlate with any change in mean CD4 count (data not shown) and is restricted by HLA-B*07 (30). Another amino acid, Asn, in the same residue is also very common (n=56), but is defined as a neutral drift by QUASI. This mutation is correlated with HLA-B*35 (P=0.004) and it has a significant change in the mean CD4 count from 280.56 to 508.62 CD4 cells/ml (P=7.31E-04). An Asn mutation may hinder the hydrophobic and van der Waals interactions when binding cyclophilin A, and consequently reduce incorporation of cypA that will affect viral fitness (70).

P6 gag is critical in incorporating the accessory protein vpr into the mature virion, and several studies have attempted to establish the region of the protein that is important in vpr binding (71-73). The (Lxx)4 region has been implicated in vpr binding (74), however mutational analysis indicates that the 462-FRFG region is most important (75). Our A clade consensus at this region is −462-FGMG, a sequence which differs notably from the aforementioned 462FRFG (a sequence which did not occur in our A clade samples). Interestingly, the mutation G465R was strongly associated with higher CD4 counts (502.74 cells/ml compared to the consensus 355.85 cells/ml), indicating that the glycine at position 465 could be functionally important. Similarly the D clade viruses (the consensus is −463-FGFG) that contain the G464R mutation is associated with an mean CD4 count of 420.82 cells/ml that is much higher than the CD4 counts for the consensus average of 292.82 cell/ml, though it was not statistically significant (P<0.15, n=98—the variances between the two were unequal, P=0.38, and this rendered the means comparison insignificant). The interaction between vpr and p6 has not been clearly established, nor has that between vpr and the nuclear pore complex. There are suggestions that vpr preferentially interacts with FG repeats in nuclear pore proteins (76), but there has not been a clear consensus reached on this topic. Our data suggests an importance for the Gly residues at these locations; however further functional analysis would need to be conducted to establish its importance in vpr interaction.

In conclusion, a comprehensive understanding of the relationship between host restricted selection and immune escape can greatly help in designing of a vaccine. Ultimately, an efficient approach to understanding viral evolution would require large-scale sequence analysis. The method that we have outlined is one such approach. Identifying positively selected amino acids and studying their relationships with the host (in the context of CTL pressure and their effect on disease progression) plays no small role in increasing our means of identifying potential CTL epitopes, and viral adaptation. With this knowledge, we can elucidate the detailed mechanisms of immune escape, and where the virus may be vulnerable. We believe that this method is a sound and productive informatic approach to studying suspected escape mutations, and the predictive method has been confirmed in in vitro functional studies.

All published documents mentioned in the above specification are herein incorporated by reference. Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Various modifications of the above-described modes for carrying out the invention which are clear to those skilled in the field of genetics and molecular biology or related fields are intended to be within the scope of the following claims.

### REFERENCES

1. UNAIDS. http://www.unaids.org. http://www.unaids.org: http://www.unaids.org.
2. Addo M M, Altfeld M, Rosenberg E S, et al. The HIV-1 regulatory proteins Tat and Rev are frequently targeted by cytotoxic T lymphocytes derived from HIV-1-infected individuals. Proc Natl Acad Sci USA 2001; 98(4):1781-6.
3. Addo M M, Yu X C, Rathod A, et al. Comprehensive epitope analysis of human immunodeficiency virus type 1 (HIV-1)-specific T-cell responses directed against the entire expressed HIV-1 genome demonstrate broadly directed responses, but no correlation to viral load. J Virol 2003; 77(3):2081-92.
4. Addo M M, Yu X G, Rosenberg E S, Walker B D, Altfeld M. Cytotoxic T-lymphocyte (CTL) responses directed against regulatory and accessory proteins in HIV-1 infection. DNA Cell Biol 2002; 21(9):671-8.
5. Allen T M, Altfeld M, Yu X G, et al. Selection, transmission, and reversion of an antigen-processing cytotoxic T-lymphocyte escape mutation in human immunodeficiency virus type 1 infection. J Virol 2004; 78(13):7069-78.
6. Altfeld N, Addo M M, Eldridge R L, et al. Vpr is preferentially targeted by CTL during HIV-1 infection. J Immunol 2001; 167(5):2743-52.
7. Altfeld M, Rosenberg E S, Shankarappa R, et al. Cellular immune responses and viral diversity in individuals treated during acute and early HIV-1 infection. J Exp Med 2001; 193(2):169-80.
8. Brander C, Walker B D. T lymphocyte responses in HIV-1 infection: implications for vaccine development. Curr Opin Immunol 1999; 11(4):451-9.
9. Cao H, Kanki P, Sankale J L, et al. Cytotoxic T-lymphocyte cross-reactivity among different human immunodeficiency virus type 1 clades: implications for vaccine development. J Virol 1997; 71(11):8615-23.
10. Cao H, Mani I, Vincent R, et al. Cellular immunity to human immunodeficiency virus type 1 (HIV-1) clades: relevance to HIV-1 vaccine trials in Uganda. J Infect Dis 2000; 182(5):1350-6.
11. Draenert R, Verrill C L, Tang Y, et al. Persistent recognition of autologous virus by high-avidity CD8 T cells in chronic, progressive human immunodeficiency virus type 1 infection. J Virol 2004; 78(2):630-41.
12. Ferrari G, Kostyu D D, Cox J, et al. Identification of highly conserved and broadly cross-reactive HIV type 1 cytotoxic T lymphocyte epitopes as candidate immunogens for inclusion in Mycobacterium bovis BCG-vectored HIV vaccines. AIDS Res Hum Retroviruses 2000; 16(14):1433-43.
13. Frahm N, Korber B T, Adams C M, et al. Consistent cytotoxic-T-lymphocyte targeting of immunodominant regions in human immunodeficiency virus across multiple ethnicities. J Virol 2004; 78(5):2187-200.
14. Galea P, le Contel C, Coutton C, Chermann J C. Rationale for a vaccine using cellular-derived epitope presented by HIV isolates. Vaccine 1999; 17(13-14):1700-5.
15. Gillespie G M, Kaul R, Dong T, et al. Cross-reactive cytotoxic T lymphocytes against a HIV-1 p24 epitope in slow progressors with B*57. Aids 2002; 16(7):961-72.
16. Goulder P J, Bunce M, Krausa P, et al. Novel, cross-restricted, conserved, and immunodominant cytotoxic T lymphocyte epitopes in slow progressors in HIV type 1 infection. AIDS Res Hum Retroviruses 1996; 12(18):1691-8.
17. Langlade-Demoyen P, Ngo-Giang-Huong N, Ferchal F, Oksenhendler E. Human immunodeficiency virus (HIV) nef-specific cytotoxic T lymphocytes in noninfected heterosexual contact of HIV-infected patients. J Clin Invest 1994; 93(3):1293-7.
18. McMichael A, Hanke T. The quest for an AIDS vaccine: is the CD8+ T-cell approach feasible? Nat Rev Immunol 2002; 2(4):283-91.
19. McMichael A J, Ogg G, Wilson J, et al. Memory CD8+ T cells in HIV infection. Philos Trans R Soc Lond B Biol Sci 2000; 355(1395):363-7.
20. Rowland-Jones S, Dong T, Krausa P, et al. The role of cytotoxic T-cells in HIV infection. Dev Biol Stand 1998; 92:209-14.
21. Rowland-Jones S, Sutton J, Ariyoshi K, et al. HIV-specific cytotoxic T-cells in HIV-exposed but uninfected Gambian women. Nat Med 1995; 1(1):59-64.
22. Rowland-Jones S, Tan R, McMichael A. Role of cellular immunity in protection against HIV infection. Adv Immunol 1997; 65:277-346.
23. Rowland-Jones S L, Dong T. Fowke K R, et al. Cytotoxic T cell responses to multiple conserved HIV epitopes in HIV-resistant prostitutes in Nairobi. J Clin Invest 1998; 102(9):1758-65.
24. Rowland-Jones S L, Nixon D F, Aldhous M C, et al. HIV-specific cytotoxic T-cell activity in an HIV-exposed but uninfected infant. Lancet 1993; 341(8849):860-1.
25. Sabbaj S, Bansal A, Ritter G D, et al. Cross-reactive CD8+ T cell epitopes identified in US adolescent minorities. J Acquir Immune Defic Syndr 2003; 33(4):426-38.
26. Yang O O, Walker B D. CD8+ cells in human immunodeficiency virus type I pathogenesis: cytolytic and noncytolytic inhibition of viral replication. Adv Immunol 1997; 66:273-311.
27. Rowland-Jones S L, Dong T, Dorrell L, et al. Broadly cross-reactive HIV-specific cytotoxic T-lymphocytes in highly-exposed persistently seronegative donors. Immunol Lett 1999; 66(1-3):9-14.
28. Rowland-Jones S L, Pinheiro S, Kaul R, et al. How important is the ‘quality’ of the cytotoxic T lymphocyte (CTL) response in protection against HIV infection? Immunol Lett 2001; 79(1-2):15-20.
29. Leslie A J, Pfafferott K J, Chetty P, et al. HIV evolution: CTL escape mutation and reversion after transmission. Nat Med 2004; 10(3):282-9.
30. Kiepiela P, Leslie A J, Honeyborne I, et al. Dominant influence of HLA-B in mediating the potential co-evolution of HIV and HLA. Nature 2004; 432(7018):769-75.
31. McMichael A J, Phillips R E. Escape of human immunodeficiency virus from immune control. Annu Rev Immunol 1997; 15:271-96.
32. McMichael A. How viruses hide from T cells. Trends Microbiol 1997; 5(6):211-2; discussion 212-3.
33. McMichael A, Klenerman P. HIV/AIDS. HLA leaves its footprints on HIV. Science 2002; 296(5572):1410-1.
34. Walker B D, Goulder P J. AIDS. Escape from the immune system. Nature 2000; 407(6802):313-4.
35. Walker B D, Korber B T. Immune control of HIV: the obstacles of HLA and viral diversity. Nat Immunol 2001; 2 (6):473-5.
36. Yang O O, Sarkis P T, All A, et al. Determinant of HIV-1 mutational escape from cytotoxic T lymphocytes. J Exp Med 2003; 197(10):1365-75.
37. Yang O O, Kalams S A, Trocha A, et al. Suppression of human immunodeficiency virus type 1 replication by CD8+ cells: evidence for HLA class I-restricted triggering of cytolytic and noncytolytic mechanisms. J Virol 1997; 71(4):3120-8.
38. Takiguchi M. [Role of HLA in HIV-1 infection]. Uirusu 2000; 50(1):47-55.
39. Stewart J J, Watts P, Litwin S. An algorithm for mapping positively selected members of quasispecies-type viruses. BMC Bioinformatics 2001; 2:1.
40. Simonsen J N, Plummer F A, Ngugi E N, et al. HIV infection among lower socioeconomic strata prostitutes in Nairobi. Aids 1990; 4(2):139-44.
41. Luo M, Blanchard J, Brunham K, et al. Two-step high resolution sequence-based HLA-DRB typing of exon 2 DNA with taxonomy-based sequence analysis allele assignment. Hum Immunol 2001; 62(11):1294-310.
42. Luo M, Blanchard J, Pan Y, Brunham K, Brunham R C. High-resolution sequence typing of HLA-DQA1 and -DQB1 exon 2 DNA with taxonomy-based sequence analysis (TBSA) allele assignment. Tissue Antigens 1999; 54(1):69-82.
43. Luo M, Embree J, Ramdahin S, et al. HLA-A and HLA-B in Kenya, Africa: allele frequencies and identification of HLA-B*1567 and HLA-B*4426. Tissue Antigens 2002; 59(5):370-80.
44. Kumar S, Tamura K, Nei M. MEGA3: Integrated software for Molecular Evolutionary Genetics Analysis and sequence alignment. Brief Bioinform 2004; 5(2):150-63.
45. HIVdatabase. HIV sequence database., 2005.
46. Korber B, Muldoon N, Theiler J, et al. Timing the ancestor of the HIV-1 pandemic strains. Science 2000; 288(5472):1789-96.
47. Nielsen M, Lundegaard C, Lund O, Kesmir C. The role of the proteasome in generating cytotoxic T-cell epitopes: insights obtained from improved predictions of proteasomal cleavage. Immunogenetics 2005; 57(1-2):33-41.
48. Kesmir C, Nussbaum A K, Schild H, Detours V, Brunak S. Prediction of proteasome cleavage motifs by neural networks. Protein Eng 2002; 15(4):287-96.
49. Peters B, Bulik S, Tampe R, Van Endert P M, Holzhutter H G. Identifying MHC class I epitopes by predicting the TAP transport efficiency of epitope precursors. J Immunol 2003; 171(4):1741-9.
50. Kelleher A D, Long C, Holmes E C, et al. Clustered mutations in HIV-1 gag are consistently required for escape from HLA-B27-restricted cytotoxic T lymphocyte responses. J Exp Med 2001; 193(3):375-86.
51. Jassoy C, Johnson R P, Navia B A, Worth J, Walker B D. Detection of a vigorous HIV-1-specific cytotoxic T lymphocyte response in cerebrospinal fluid from infected persons with AIDS dementia complex. J Immunol 1992; 149(9):3113-9.
52. Leslie A, Kavanagh D, Honeyborne I, et al. Transmission and accumulation of CTL escape variants drive negative associations between HIV polymorphisms and HLA. J Exp Med 2005; 201(6):891-902.
53. Martinez-Picado J, Prado J G, Fry E E, et al. Fitness cost of escape mutations in p24 Gag in association with control of human immunodeficiency virus type 1. J Virol 2006; 80(7):3617-23.
54. Migueles S A, Laborico A C, Imamichi H, et al. The differential ability of HLA B*5701+ long-term nonprogressors and progressors to restrict human immunodeficiency virus replication is not caused by loss of recognition of autologous viral gag sequences. J Virol 2003; 77(12):6889-98.
55. Stewart-Jones G B, Gillespie G, Overton I M, et al. Structures of three HIV-1 HLA-B*5703-peptide complexes and identification of related HLAs potentially associated with long-term nonprogression. J Immunol 2005; 175(4):2459-68.
56. Rammensee H, Bachmann J, Emmerich N P, Bachor O A, Stevanovic S. SYFPEITHI: database for MHC ligands and peptide motifs. Immunogenetics 1999; 50(3-4):213-9.
57. Craiu A, Akopian T, Goldberg A, Rock K L. Two distinct proteolytic processes in the generation of a major histocompatibility complex class I-presented peptide. Proc Natl Acad Sci USA 1997; 94(20):10850-5.
58. Serwold T, Gaw S, Shastri N. ER aminopeptidases generate a unique pool of peptides for MHC class I molecules. Nat Immunol 2001; 2(7):644-51.
59. Draenert R, Le Gall S, Pfafferott K J, et al. Immune selection for altered antigen processing leads to cytotoxic T lymphocyte escape in chronic HIV-1 infection. J Exp Med 2004; 199(7):905-15.
60. Goulder P J, Brander C, Annamalai K, et al. Differential narrow focusing of immunodominant human immunodeficiency virus gag-specific cytotoxic T-lymphocyte responses in infected African and caucasoid adults and children. J Virol 2000; 74(12):5679-90.
61. Dawson L, Yu X F. The role of nucleocapsid of HIV-1 in virus assembly. Virology 1998; 251(1):141-57.
62. Schmalzbauer E, Strack B, Dannull J, Guehmann S, Moelling K. Mutations of basic amino acids of NCp7 of human immunodeficiency virus type 1 affect RNA binding in vitro. J Virol 1996; 70(2):771-7.
63. Yu X G, Shang H, Addo M M, et al. Important contribution of p15 Gag-specific responses to the total Gag-specific CTL responses. Aids 2002; 16(3):321-8.
64. Mo X Y, Cascio P, Lemerise K, Goldberg A L, Rock K. Distinct proteolytic processes generate the C and N termini of MHC class I-binding peptides. J Immunol 1999; 163(11):5851-9.
65. Paz P, Brouwenstijn N, Perry R, Shastri N. Discrete proteolytic intermediates in the MHC class I antigen processing pathway and MHC I-dependent peptide trimming in the ER. Immunity 1999; 11(2):241-51.
66. Stoltze L, Dick T P, Deeg M, Pommerl B, Rammensee H G, Schild H. Generation of the vesicular stomatitis virus nucleoprotein cytotoxic T lymphocyte epitope requires proteasome-dependent and -independent proteolytic activities. Eur J Immunol 1998; 28(12):4029-36.
67. Larsen M V, Lundegaard C, Lamberth K, et al. An integrative approach to CTL epitope prediction: a combined algorithm integrating MHC class I binding, TAP transport efficiency, and proteasomal cleavage predictions. Eur J Immunol 2005; 35(8):2295-303.
68. Barber L D, Percival L, Arnett K L, Gumperz J E, Chen L, Parham P. Polymorphism in the alpha 1 helix of the HLA-B heavy chain can have an overriding influence on peptide-binding specificity. J Immunol 1997; 158(4):1660-9.
69. Friedrich T C, Dodds E J, Yant L J, et al. Reversion of CTL escape-variant immunodeficiency viruses in vivo. Nat Med 2004; 10(3):275-81.
70. Gamble T R, Vajdos F F, Yoo S, et al. Crystal structure of human cyclophilin A bound to the amino-terminal domain of HIV-1 capsid. Cell 1996; 87(7):1285-94.
71. Holguin A, Alvarez A, Soriano V. Variability in the P6gag domains of HIV-1 involved in viral budding. Aids 2006; 20(4):624-7.
72. Demirov D G, Orenstein J M, Freed E O. The late domain of human immunodeficiency virus type 1 p6 promotes virus release in a cell type-dependent manner. J Virol 2002; 76(1):105-17.
73. Demirov D G, Ono A, Orenstein J M, Freed E O. Overexpression of the N-terminal domain of TSG101 inhibits HIV-1 budding by blocking late domain function. Proc Natl Acad Sci USA 2002; 99(2):955-60.
74. Kondo E, Gottlinger H G. A conserved LXXLF sequence is the major determinant in p6gag required for the incorporation of human immunodeficiency virus type 1 Vpr. J Virol 1996; 70(1):159-64.
75. Zhu H, Jian H, Zhao L J. Identification of the 15FRFG domain in HIV-1 Gag p6 essential for Vpr packaging into the virion. Retrovirology 2004; 1(1):26.
76. Fouchier R A, Meyer B E, Simon J H, et al. Interaction of the human immunodeficiency virus type 1 Vpr protein with the nuclear pore complex. J Virol 1998; 72(7):6004-13.
77. Altman J D, et al. Phenotypic analysis of antigen-specific T lymphocytes. Science, 1996. 274(5284):94-6.
78. Schmittel A, et al. Evaluation of the interferon-gamma ELISPOT-assay for quantification of peptide specific T lymphocytes from peripheral blood. J. Immunol. Methods, 1997. 210(2):167-74.

TABLE 1
HLA and CD4 correlations to positively selected amino acids in clade
A1 gag proteins.

| | | | Correlation^{a} | | Δ CD4 | |
|---|---|---|---|---|---|---|
| gag | Mutation | HLA Allele | (p-value) | | (p-value)^{b} | |
| p17 | V7I | B*5801 | + | 7.61E−07 | n/a | |
| | K12Q | A*74 | + | 7.77E−11 | n/a | |
| | D14E | A*74 | + | 9.19E−08 | n/a | |
| | R20Q | A*74 | + | 7.04E−09 | n/a | |
| | R20Q | B*3501 | + | 2.17E−05 | n/a | |
| | K26R | C*06 | + | 8.25E−05 | n/a | |
| | K28Q | A*0301 | + | 1.65E−16 | n/a | |
| | K28Q | A*3001 | + | 5.96E−06 | n/a | |
| | R30K | A*2301 | − | 7.47E−03 | n/a | |
| | S38G | A*01 | + | 5.48E−05 | n/a | |
| | E42D | B*3502 | + | 1.96E−04 | −75.25 | 1.16E−02 |
| | R43K | B*1510 | + | 5.75E−05 | n/a | |
| | R43K | C*0304 | + | 1.01E−05 | n/a | |
| | S49G | B*1402 | + | 8.30E−05 | n/a | |
| | P66S | A*0202 | − | 2.54E−03 | n/a | |
| | A67S | C*0701 | + | 1.39E−05 | −124.01 | 1.49E−02 |
| | L68I | B*4102 | + | 1.50E−05 | −93.69 | 3.34E−02 |
| | L75I | A*0201 | + | 2.85E−03 | n/a | |
| | L75I | A*0202 | + | 8.16E−07 | n/a | |
| | L75I | A*02 | + | 1.68E−05 | n/a | |
| | F79Y | A*0101 | − | 4.02E−05 | n/a | |
| | F79Y | A*0202 | + | 2.26E−05 | n/a | |
| | F79Y | A*02 | + | 5.56E−04 | n/a | |
| | F79Y | A*3002 | − | 8.38E−05 | n/a | |
| | F79Y | A*36 | − | 1.55E−04 | n/a | |
| | F79Y | B*5703 | + | 5.67E−03 | n/a | |
| | T81A | A*0201 | − | 4.08E−03 | n/a | |
| | T81A | A*3009 | + | 2.17E−03 | n/a | |
| | T81A | B*5801 | − | 2.27E−04 | n/a | |
| | T81A | B*5802 | + | 8.31E−03 | n/a | |
| | V82I | A*0204 | + | 7.86E−03 | −89.08 | 7.30E−03 |
| | V82I | A*0240 | + | 1.57E−05 | | |
| | L85I | A*0201 | + | 1.83E−03 | n/a | |
| | L85I | A*0240 | + | 1.25E−04 | n/a | |
| | L85I | A*02 | + | 4.61E−03 | n/a | |
| | V88I | A*0240 | + | 3.17E−07 | n/a | |
| | V88I | B*4504 | + | 2.00E−06 | n/a | |
| | V88I | C*0606 | + | 2.78E−09 | n/a | |
| | V88I | C*0722 | + | 2.78E−09 | n/a | |
| | V88I | C*1601 | + | 2.24E−05 | n/a | |
| | I92M | A*0204 | + | 4.23E−09 | n/a | |
| | I92M | A*02 | + | 3.12E−03 | n/a | |
| | I92M | B*51 | + | 7.63E−05 | n/a | |
| | D93E | C*06 | − | 5.03E−08 | n/a | |
| | S111N | B*51 | + | 5.82E−06 | n/a | |
| | T115A | A*3001 | + | 7.32E−11 | n/a | |
| P24 | V158A | A*0201 | + | 2.85E−03 | n/a | |
| | I159V | B*5703 | + | 2.46E−03 | n/a | |
| | A163G | B*5703 | + | 1.96E−10 | 88.04 | 3.77E−02 |
| | A163G | B*57 | + | 1.40E−07 | | |
| | I190V | B*5802 | + | 1.36E−04 | −92.31 | 1.76E−02 |
| | I190V | C*07 | − | 2.03E−03 | | |
| | V218A | B*3501 | + | 1.00E−02 | −138.04 | 2.58E−04 |
| | P243T | B*5702 | + | 8.19E−04 | n/a | |
| | P243T | B*5703 | + | 2.07E−03 | n/a | |
| | P243T | B*57 | + | 1.26E−06 | n/a | |
| | I247L | B*5702 | + | 3.02E−05 | n/a | |
| | I247L | B*57 | + | 1.00E−04 | n/a | |
| | I247L | C*1801/02 | + | 6.32E−05 | n/a | |
| | T303A/I | B*1510 | + | 4.49E−03 | n/a | |
| | T303A/I | C*0304 | + | 2.53E−06 | n/a | |
| | T310S | B*4901 | + | 2.58E−08 | n/a | |
| | T310S | C*0407 | + | 7.85E−05 | n/a | |
| | E312D | B*45 | + | 2.78E−05 | n/a | |
| | E312D | B*4901 | + | 2.02E−06 | n/a | |
| | E312D | B*5802 | − | 4.11E−03 | n/a | |
| | E319D | B*4415 | + | 7.30E−03 | n/a | |
| | E319D | B*4501 | + | 7.61E−06 | n/a | |
| | T342S | C*0804 | + | 1.50E−05 | n/a | |
| | G357S | B*07 | − | 7.31E−03 | n/a | |
| | G357S | C*1601 | + | 7.99E−07 | n/a | |
| p7 | R384K | A*7401/02 | + | 9.00E−03 | −70.21 | 1.60E−02 |
| | R384K | C*0407 | + | 3.30E−02 | | |
| | K387R | A*7401/02 | + | 3.53E−05 | n/a | |
| | R402K | B*1405 | + | 4.00E−03 | n/a | |
| p1 | K435R | B*1302 | + | 6.83E−05 | n/a | |
| p6 | G465R | | | | 146.89 | 4.00E−03 |
| | K489R | A*3601 | + | 8.00E−05 | n/a | |
| | K489R | B*1801 | + | 8.60E−06 | n/a | |
| | G493S | A*0240 | + | 2.38E−06 | n/a | |
| | P496L | B*5703 | − | 3.00E−03 | n/a | |

^{a}Positive correlations are shown as (+) and negative correlations (−).
^{b}CD4 data are shown for those CD4 (cells/ml) with significant changes.
^{c}“Undocumented” refers to potential novel epitopes. “Reference” denotes previously defined epitopes.

TABLE 2
HLA and CD4 correlations to positively selected amino acids in clade
D gag proteins.

| | | | Correlation^{a} | | | |
|---|---|---|---|---|---|---|
| gag | Mutation | HLA-Allele | (p-value) | | Δ CD4 (p-value)^{b} | |
| p17 | L8I | C*0701 | − | 1.86E−09 | n/a | |
| | G11E | A*03 | + | 4.13E−07 | n/a | |
| | K28R | A*3001 | + | 3.66E−04 | n/a | |
| | K30R | A*2301/07N | + | 7.59E−04 | n/a | |
| | K30R | A*23 | + | 5.96E−03 | n/a | |
| | K30R | A*24 | + | 6.53E−03 | n/a | |
| | G62E/A | B*53 | + | 8.26E−05 | −140.79 | 1.13E−03 |
| | S72T | B*1503 | − | 1.76E−06 | n/a | |
| | E74G | B*44 | + | 6.35E−05 | n/a | |
| | I75L | A*02 | + | 2.79E−02 | n/a | |
| | Y79F | A*0101 | + | 3.89E−02 | 125.12 | 1.70E−02 |
| | Y79F | A*3002 | + | 2.54E−02 | | |
| | S125N | B*3501 | + | 5.03E−05 | n/a | |
| p24 | A146P | B*5703 | + | 1.63E−07 | n/a | |
| | A146P | B*57 | + | 3.69E−10 | n/a | |
| | A146P | B*58 | − | 5.23E−03 | n/a | |
| | I147L | B*57 | + | 3.21E−03 | | |
| | I147L | B*5801 | + | 9.61E−03 | −72.83 | 1.38E−02 |
| | I147L | B*5802 | − | 7.29E−03 | | |
| | T242N | B*5703 | + | 1.63E−07 | n/a | |
| | T242N | B*57 | + | 3.69E−10 | n/a | |
| | T242N | B*5801 | + | 2.74E−09 | n/a | |
| | I256V | A*0201 | + | 1.82E−03 | n/a | |
| | S310T | B*5301 | + | 3.40E−03 | n/a | |
| | S310T | C*0401 | + | 3.21E−03 | n/a | |
| | N315T | B*4415 | + | 9.54E−14 | n/a | |
| | V323I | B*51 | + | 2.08E−08 | −125.93 | 1.62E−02 |
| | T342S | B*5703 | + | 8.50E−05 | n/a | |
| | S357G | B*0702 | + | 1.29E−04 | n/a | |
| p7 | K406R | A*0301 | + | 1.00E−03 | n/a | |
| | K414R | | | | −124.68 | 2.40E−02 |
| | K421R | | | | 137.57 | 2.20E−02 |
| p1 | K436R | B*1302 | + | 6.83E−05 | n/a | |
| | S440P | | | | 164.47 | 1.20E−02 |
| p6 | G464R | B*4016 | + | 1.85E−05 | n/a | |
| | S487A | A*6802 | + | 8.99E−09 | n/a | |
| | S497L | | | | 87.81 | 3.50E−02 |

^{a}Positive correlations are shown as (+) and negative correlations (−).
^{b}CD4 data are shown for those CD4 (cells/ml) with significant changes.
^{c}“Undocumented” refers to potential novel epitopes. “Reference” denotes previously identified epitopes.

TABLE 3
Frequency of positive and negative HLA correlations to positively selected
amino acids at P < 0.01.

| | Clade A1 | | | | |
|---|---|---|---|---|---|
| | +corre- | | Clade D | | Undefined |
| gag | lation | −correlation | +correlation | −correlation | epitopes^{a} |
| p17 | 39 | 8 | 11 | 2 | 32 |
| p24 | 21 | 3 | 14 | 2 | 8 |
| p7 | 4 | 0 | 1 | 0 | 1 |
| p1 | 1 | 0 | 1 | 0 | 1 |
| p6 | 3 | 1 | 2 | 0 | 5 |

^{a}HLA correlations that have not been determined in previous studies.

TABLE 4

Deduced peptide sequences of undocumented
epitopes

| | | | | Position |
|---|---|---|---|---|
| | Positive/ | | | of 1^{st} |
| | negative | | | amino |
| Peptide | selected | | Gag | acids |
| sequences | mutation | HLA allele | protein | in gag |
| SVLSGGKLD | V7I | B*5801 | p17 | 6 |
| SILSGGKLD | | | | |
| RASVLSGGK | K12Q | A*7401/02 | p17 | 4 |
| RASVLSGGQ | | | | |
| ASVLSGGKL | | | | 5 |
| ASVLSGGQL | | | | |
| GKLDAWEKI | 11 | | | |
| GQLDAWEKI | | | | |
| SVLSGGKLD | D14E | A*7401/02 | p17 | 6 |
| SVLSGGKLE | | | | |
| VLSGGKLDA | | | | 7 |
| VLSGGKLEA | | | | |
| LDAWEKIRL | | | | 13 |
| LEAWEKIRL | | | | |
| ELDAWEKIR | R20Q | A*7401/02 | p17 | 12 |
| KLDAWEKIQ | | | | |
| LDAWEKIRL | | B*3501 | | 13 |
| LEAWEKIRL | | | | |
| LDAWEKIQL | | | | |
| LEAWEKIQL | | | | |
| IRLRPGGKK | | | | 19 |
| IQLRPGGKK | | | | |
| KIRLRPGGK | K26R | C*0602 | p17 | 18 |
| KIRLRPGGR | | | | |
| IRLRPGGKI | | | | 19 |
| IRLRPGGRK | | | | |
| GKKKYRMKH | | | | 25 |
| GRKKYRMKH | | | | |
| RNKHLVWAS | S38G | A*0101 | p17 | 30 |
| RMKHLVWAG | | | | |
| MKHLVWASR | | | | 31 |
| MKHLVWAGR | | | | |
| ASRELERFA | | | | 37 |
| AGRELERFA | | | | |
| VWASRELER | R43K | B*1510 | p17 | 35 |
| VWASRELEK | | C*0304 | | |
| WASRELERF | | | | 36 |
| WASRELEKF | | | | |
| ERFALNPSL | | | | 42 |
| EKFALNPSL | | | | |
| LERFALNPS | S49G | B*1402 | p17 | 41 |
| LERFALNPG | | | | |
| ERFALNPSL | | | | 42 |
| ERFALNPGL | | | | |
| PSLLETTEG | | | | 48 |
| PGLLETTEG | | | | |
| QQIMEQLQP | P66S | NEG | p17 | 58 |
| QQIMEQLQS | | A*0202 | | |
| QIMEQLQPA | | | | 59 |
| QIMEQLQSA | | | | |
| QPALKTGTE | | | | 65 |
| QSALKTGTE | | | | |
| QIMEQLQPA | A67S | C*0701 | p17 | 59 |
| QIMEQLQPS | | | | |
| IMEQLQPAL | | | | 60 |
| IMEQLQPSL | | | | |
| PALKTGTEE | | | | 66 |
| PSLKTGTEE | | | | |
| EELRSLFNT | T81A | NEG | p17 | 73 |
| EELRSLFNA | | B*5801 | | |
| ELRSLFNTV | | POS | | 74 |
| ELRSLFNAV | | B*5802 | | |
| NTVATLYCV | | | | 80 |
| NAVATLYCV | | | | |
| NTVATLYCV | V88I | C*1601 | P17 | 80 |
| NTVATLYCI | | | | |
| TVATLYCVH | | | | 81 |
| TVATLYCIH | | | | |
| CVHQRIDVK | | | | 87 |
| CIHQRIDVK | | | | |
| TLYCVHQRI | I92M | A*0202 | p17 | 84 |
| TLYCVHQRM | | | | |
| LYCVHQRID | | | | 85 |
| LYCVHQRMD | | | | |
| RIDVKDTKE | | | | 91 |
| RMDVKDTKE | | | | |
| LYCVHQRID | D93E | NEG | p17 | 85 |
| LYCVHQRIE | | C*0602 | | |
| YCVHQRIDV | | | | 86 |
| YCVHQRIEV | | | | |
| IDVKDTKEA | | | | 92 |
| IEVKDTKEA | | | | |
| IQNKSKQKT | T115A/K/P | A*3001 | p17 | 107 |
| IQNKSKQKA | | | | |
| IQWKSKQKK | | | | |
| IQNKSKQKP | | | | |
| QNKSKQKTQ | | | | 108 |
| QNKSKQKAQ | | | | |
| QNKSKQKKQ | | | | |
| QNKSKQKPQ | | | | |
| KTQQAAADT | | | | 114 |
| KAQQAAADT | | | | |
| KKQQAAADT | | | | |
| KPQQAAADT | | | | |
| TTSTPQEQI | I247L | C*0801/02 | p24 | 107 |
| TTSTPQEQL | | | | |
| TSTPQEQIG | | | | 108 |
| TSTPQEQLG | | | | |
| QIGWMTSNP | | | | 114 |
| QLGWMTSNP | | | | |
| KTLRAEQAT | T130S | B*4901 | p24 | 170 |
| KTLRAEQAS | | | | |
| TLRAEQATQ | | | | 171 |
| TLRAEQASQ | | | | |
| ATQEVKGWM | | | | 177 |
| ATQDVKGWM | | | | |
| ASQEVKGWM | | | | |
| ASQDVKGWM | | | | |
| LRAEQATQE | E312D | B*4901 | p24 | 172 |
| LRAEQASQE | | | | |
| LRAEQATQD | | | | |
| LRAEQASQD | | | | |
| RAEQATQEV | | | | 173 |
| RAEQATQDV | | | | |
| QEVKGWMTE | | | | 179 |
| QDVKGWMTE | | | | |
| QEVKGWMTE | E319D | B*4501 | p24 | 311 |
| QEVKGWNTD | | | | |
| EVKGWMTET | | | | 312 |
| EVKGWMTDT | | | | |
| TETLLVQNA | | | | 318 |
| TDTLLIQNA | | | | |
| LRALGTGAT | T342S | C*0804 | p24 | 334 |
| LRALGTGAS | | | | |
| RALGTGATL | | | | 335 |
| RALGTGASL | | | | |
| ATLEEMMTA | | | | 341 |
| ASLEEMMTA | | | | |
| LKALGPAAT | T342S | B*5703 | p24 | 334 |
| LKALGPAAS | | | | |
| KALGPAATL | | | | 335 |
| KALGPAASL | | | | |
| ATLEEMMTA | | | | 341 |
| ASLEEMMTA | | | | |
| ACQGVGGPG | G357S | C*1601 | p24 | 349 |
| ACQCVGGPS | | | | |
| CQGVGGPGH | | | | 350 |
| CQGVGGPSH | | | | |
| QRGNFRGQK | K387R | A*7401/02 | p7 | 379 |
| QRGNFRGQR | | | | |
| RGNFKGPKK | | | | 380 |
| RGNFKGPRK | | | | |
| QKRIKCFNC | | | | 386 |
| QRRIKCFNC | | | | |
| ERQANFLGK | K435R | B*1302 | p7-p1 | 427 |
| ERQANFLGR | | | | |
| RQANFLGKI | | | | 428 |
| RQANWLGRI | | | | |
| GKIWPSSKG | | | | 434 |
| GRIWPSSKG | | | | |
| TAPPAESFC | G464R | B*4016 | p6 | 456 |
| TAPPAESFR | | | | |
| APPAESFGF | | | | 457 |
| APPAESFRF | | | | |
| FGFGEEITS | | | | 463 |
| FRFGEEITS | | | | |
| DKELYPLAS | S487A | A*6802 | p6 | 479 |
| DKELYPLAA | | | | |
| KELYPLASL | | | | 480 |
| KELYPLAAL | | | | |
| ASLKSLFGS | | | | 486 |
| AALKSLFGS | | | | |
| QAPPLVSLK | K489R | A*3601 | p6 | 481 |
| QAPPLVSLR | | B*1801 | | |
| ASPLVSLKS | | | | 482 |
| ASPLVSLRS | | | | |
| LKSLFGNDL | | | | 488 |
| LRSLFGNDL | | | | |
| LVSLKSLFG | G493S | A*0240 | p6 | 485 |
| LVSLKSLFS | | | | |
| VSLKSLFGN | | | | 486 |
| VSLKSLFSN | | | | |

TABLE 5

TAP affinity score for RK9, SL9, MV9,
and LARK10 of gag.

| | | | | |
|---|---|---|---|---|
| Clade | gag | Epitope | Mutation(S)^{a} | Log IC₅₀ (nM)^{b} |

| | | | | |
|---|---|---|---|---|
| A1 | p17 | RK9 | RLRPGGKKK | −2.12 |
| | | | RLRPGGKKQ | −1.55 |
| | | | QLRPGGKKK | −0.31 |
| | | | QLRPGGKKQ | 0.26 |
| | | SL9 | SLFNTVATL | −2.46 |
| | | | SLYNTVATL | −3.21 |
| D | p24 | MV9 | MTSNPPIPV | −0.61 |
| | | | MTNNPPIPV | 0.29 |
| | | | LARNCRAPRK | −1.62 |
| | | | IAKNCRAPRK | 0.09 |
| | | | IARNCRAPRK | −1.23 |

^{a}Sites of variants are bolded each with the corresponding TAP score.
^{b}Log IC₅₀ (nM) was calculated using t-score = mat_{1,N1} + mat_{2,N2} + mat_{3,N3} + mat_{9,C} (48).

TABLE 6
Chi-Square Analysis of HLA-B*1302 with positive selected
amino acid at position 4 of the p1 spacer protein
Crosstab

| | | | B*1302 | | |
|---|---|---|---|---|---|
| | | | 0 | 1 | Total |
| 4th aa | any positive | Count | 78 | 12 | 90 |
| re-grouping | selection | Expected Count | 87.0 | 3.0 | 90.0 |
| | other | Count | 332 | 2 | 334 |
| | | Expected Count | 323.0 | 11.0 | 334.0 |
| Total | | Count | 410 | 14 | 424 |
| | | Expected Count | 410.0 | 14.0 | 424.0 |

Chi-Square Tests

| | | | Asymp. | | |
|---|---|---|---|---|---|
| | | | Sig. | Exact Sig. | Exact Sig. |
| | Value | df | (2-sided) | (2-sided) | (1-sided) |
| Pearson Chi-Square | 36.009^{b} | 1 | .000 | | |
| Continuity | 32.131 | 1 | .000 | | |
| Correction^{a} | | | | | |
| Likelihood Ratio | 27.890 | 1 | .000 | | |
| Fisher's Exact Test | | | | .000 | .000 |
| Linear-by-Linear | 35.924 | 1 | .000 | | |
| Association | | | | | |
| N of Valid Cases | 424 | | | | |

^{a}Computed only for a 2 × 2 table
^{b}1 cells (25.0%) have expected count less than 5. The minimum expected count is 2.97.

TABLE 7
Chi-Square Analysis of HLA-B*1302 with positive selected
amino acid at position 5 of the p1 spacer protein
Crosstab

| | | | B*1302 | | |
|---|---|---|---|---|---|
| | | | 0 | 1 | Total |
| 5th aa | any positive | Count | 15 | 6 | 21 |
| re-grouping | selection | Expected Count | 20.3 | .7 | 21.0 |
| | other | Count | 395 | 8 | 403 |
| | | Expected Count | 389.7 | 13.3 | 403.0 |
| Total | | Count | 410 | 14 | 424 |
| | | Expected Count | 410.0 | 14.0 | 424.0 |

Chi-Square Tests

| | | | Asymp. | | |
|---|---|---|---|---|---|
| | | | Sig. | Exact Sig. | Exact Sig. |
| | Value | df | (2-sided) | (2-sided) | (1-sided) |
| Pearson Chi-Square | 44.187^{b} | 1 | .000 | | |
| Continuity | 36.253 | 1 | .000 | | |
| Correction^{a} | | | | | |
| Likelihood Ratio | 19.352 | 1 | .000 | | |
| Fisher's Exact Test | | | | .000 | .000 |
| Linear-by-Linear | 44.083 | 1 | .000 | | |
| Association | | | | | |
| N of Valid Cases | 424 | | | | |

^{a}Computed only for a 2 × 2 table
^{b}1 cells (25.0%) have expected count less than 5. The minimum expected count is .69.

TABLE 8

Summary of available peptides and responses to
B*1302 patient samples

| | | Patients | Patients |
|---|---|---|---|
| Peptide # | Peptide | tested | Responded |
| 40 | RQANFLGKI--- | 8 | 7 |
| 41 | RQANFLGRI--- | 8 | 7 |
| 36 | -QANFLGKIW | 6 | 0 |
| 37 | -QANFLGKLW | 6 | 0 |
| 1 | --ANFLGKIWP | 1 | 0 |
| 2 | --ANFLGKIWS | 0 | 0 |
| 26 | ---NFLGKIWPP---- | 0 | 0 |
| 27 | ---NFLGKIWPS---- | 0 | 0 |
| 28 | ---NFLGKIWSS---- | 0 | 0 |
| 29 | ---NFLGKLWPS | 0 | 0 |
| 3 | ----FLGKIWPPN | 1 | 1 |
| 4 | ----FLGKIWPPS | 4 | 1 |
| 8 | ----FLGKIWSSN | 3 | 0 |
| 5 | ----FLGKIWPSH | 3 | 1 |
| 6 | ----FLGKIWPSN | 3 | 1 |
| 7 | ----FLGKIWPSS | 4 | 1 |
| 17 | -----LGKIWPPSK | 3 | 2 |
| 18 | -----LGKIWPSHK | 3 | 1 |
| 21 | -----LGKIWSSNK | 9 | 1 |
| 20 | -----LGKIWPSSK | 10 | 4 |
| 22 | -----LGKLWPSSK | 3 | 0 |
| 23 | -----LGRIWPPSK | 3 | 1 |
| 9 | ------GKIWPSNKG | 3 | 0 |
| 10 | ------GKIWPSSKG | 8 | 2 |
| 11 | ------GRIWPSNKG | 8 | 2 |
| 38 | -------RIWPSNKGR | 1 | 0 |
| 39 | -------RIWPSSKGR | 0 | 0 |
| 15 | -------KIWSSNKGR | 3 | 0 |
| 14 | -------KIWPSSKGR | 7 | 4 |
| 16 | -------KLWPSNKGR | 7 | 4 |
| 25 | --------LWPSSKGRP | 1 | 0 |
| 24 | --------LWPPSKGRP | 0 | 0 |
| 13 | --------IWSSNKGRP | 2 | 1 |
| 12 | --------IWPSNKGRP | 2 | 0 |
| 45 | ---------WPPNKGRPG | 0 | 0 |
| 46 | ---------WPSNKGRPG | 0 | 0 |
| 48 | ---------WSSNKGRPG | 0 | 0 |
| 47 | ---------WPSSKGRPG | 0 | 0 |
| 32 | ----------PSHKGRPGN | 0 | 0 |
| 30 | ----------PPNKGRPGN | 1 | 1 |
| 31 | ----------PPSKGRPGN | 1 | 0 |
| 34 | ----------PSNKGRPGN | 0 | 0 |
| 35 | ----------PSSKGRPGN | 2 | 1 |
| 42 | -----------SHKGRPGNF | 1 | 1 |
| 43 | -----------SNKGRPGNF | [symbol] | 3 |
| 44 | -----------SSKGRPGNF | [symbol] | 2 |
| 33 | -----------PSKGRPGNF | 0 | 0 |

TABLE 9
HLA class I data for patients tested by ELISPOT against peptides
RQANFLGKI (40) and RQANFLGRI (41)

| Patient | | | | | | |
|---|---|---|---|---|---|---|
| ML # | HLA-A | | HLA-B | | HLA-C | |
| 415 | 3002 | 3402 | [1302] | 5301 | 04010101 | 0804 |
| 915 | 0103 | 2301 | [1302] | 5801 | 0602 | 070101 |
| 1160 | 3402 | 3402 | [1302] | 440301 | 04010101 | 0804 |
| 1720 | 29010101 | 3010 | 0705 | [1302] | 0602 | 150501 |
| 1728¹ | 24020101 | 3004 | [1302] | 5301 | 0602 | 0602 |
| 1778 | 0202 | 6802 | 570301 | [1302] | 0602 | 070101 |
| 1787 | 3001 | 6802 | 4201 | [1302] | 0602 | 1701 |
| 1937 | 290201 | 3402 | [1302] | 1503 | 020204 | 0602 |

¹[ ] did not respond

TABLE 10
HLA class I data for patients tested by ELISPOT against peptides
KIWPSSKGR (14) and KLWPSNKGR (16)

| Patient | | | | | | |
|---|---|---|---|---|---|---|
| ML # | HLA-A | | HLA-B | | HLA-C | |
| 57 | 0109 | 7401 | [1301] | 3537 | 04010101 | 0602 |
| [symbol] | 3002 | 3402 | [1302] | 5301 | 04010101 | 0804 |
| [symbol] | 0103 | 2301 | [1302] | 5801 | 0602 | 070101 |
| 1160 | 3402 | 3402 | [1302] | 440301 | 04010101 | 0804 |
| [symbol] | 29010101 | 3010 | 0705 | [1302] | 0602 | 150501 |
| 1728¹ | 24020101 | 3004 | [1302] | 5301 | 0602 | 0602 |
| [symbol] | 0202 | 6802 | 570301 | [1302] | 0602 | 070101 |

¹[ ] did not respond

TABLE 11
HLA data for patients tested by ELISPOT against peptides
SNKGRPGNF (43) and SSKGRPGNF (44)

| Patient | | | | | | |
|---|---|---|---|---|---|---|
| ML # | HLA-A | | HLA-B | | HLA-C | |
| 415 | 3002 | 3402 | [1302] | 5301 | 04010101 | 0804 |
| 915¹ | 0103 | 2301 | [1302] | 5801 | 0602 | 070101 |
| 1128 | 03010101 | 290201 | [1302] | 4703 | 0602 | 070101 |
| 1720 | 29010101 | 3010 | 0705 | [1302] | 0602 | 150501 |
| 1728 | 24020101 | 3004 | [1302] | 5301 | 0602 | 0602 |
| 1787 | 3001 | 6802 | 4201 | [1302] | 0602 | 1701 |
| 1937¹ | 290201 | 3402 | [1302] | 1503 | 020204 | 0602 |

¹[ ] did not respond

TABLE 12
HLA data for patients tested by ELISPOT
against peptide LGKIWPSSK (20)

| Pa- | | | | | | |
|---|---|---|---|---|---|---|
| tient | | | | | | |
| ML # | HLA-A | | HLA-B | | HLA-C | |
| 57¹ | 0109 | 7401 | [1301] | 3537 | 04010101 | 0602 |
| 415 | 3002 | 3402 | [1302] | 5301 | 04010101 | 0804 |
| 915 | 0103 | 2301 | [1302] | 5801 | 0602 | 070101 |
| 1128 | 03010101 | 290201 | [1302] | 4703 | 0602 | 070101 |
| 1160¹ | 3402 | 3402 | [1302] | 440301 | 04010101 | 0804 |
| 1720 | 29010101 | 3010 | 0705 | [1302] | 0602 | 150501 |
| 1728¹ | 24020101 | 3004 | [1302] | 5301 | 0602 | 0602 |
| 1778¹ | 0202 | 6802 | 570301 | [1302] | 0602 | 070101 |
| 1787¹ | 3001 | 6802 | 4201 | [1302] | 0602 | 1701 |
| 1937¹ | 290201 | 3402 | [1302] | 1503 | 020204 | 0602 |

¹[ ] did not respond

TABLE 13
Chi-Square Analysis of HLA-A*30 with positive selected
amino acid at position 7 of the p1 spacer protein
Crosstab

| | | | A*30 genotype | | |
|---|---|---|---|---|---|
| | | | .00 | 1.00 | Total |
| SITE7C_1 | .00 | Count | 239 | 124 | 363 |
| | | Expected Count | 235.5 | 127.5 | 363.0 |
| | 1.00 | Count | 1 | 6 | 7 |
| | | Expected Count | 4.5 | 2.5 | 7.0 |
| Total | | Count | 240 | 130 | 370 |
| | | Expected Count | 240.0 | 130.0 | 370.0 |

Chi-Square Tests

| | | | Asymp. | | |
|---|---|---|---|---|---|
| | | | Sig. | Exact Sig. | Exact Sig. |
| | Value | df | (2-sided) | (2-sided) | (1-sided) |
| Pearson Chi-Square | 8.009^{b} | 1 | .005 | | |
| Continuity | 5.907 | 1 | .015 | | |
| Correction^{a} | | | | | |
| Likelihood Ratio | 7.828 | 1 | .005 | | |
| Fisher's Exact Test | | | | .009 | .009 |
| Linear-by-Linear | 7.987 | 1 | .005 | | |
| Association | | | | | |
| N of Valid Cases | 370 | | | | |

^{a}Computed only for a 2 × 2 table
^{b}2 cells (50.0%) have expected count less than 5. The minimum expected count is 2.46.

TABLE 14
Chi-Square Analysis of HLA-A*7401/02 with positive
selected amino acid at position 9 of the p1 spacer protein
Crosstab

| | | | A*7401/02 genotype | | |
|---|---|---|---|---|---|
| | | | | 7401/02 | |
| | | | no 7401/02 | genotype | Total |
| SITE9C_1 | .00 | Count | 252 | 25 | 277 |
| | | Expected Count | 241.8 | 35.2 | 277.0 |
| | 1.00 | Count | 71 | 22 | 93 |
| | | Expected Count | 81.2 | 11.8 | 93.0 |
| Total | | Count | 323 | 47 | 370 |
| | | Expected Count | 323.0 | 47.0 | 370.0 |

Chi-Square Tests

| | | | Asymp. | | |
|---|---|---|---|---|---|
| | | | Sig. | Exact Sig. | Exact Sig. |
| | Value | df | (2-sided) | (2-sided) | (1-sided) |
| Pearson Chi-Square | 13.440^{b} | 1 | .000 | | |
| Continuity | 12.153 | 1 | .000 | | |
| Correction^{a} | | | | | |
| Likelihood Ratio | 12.028 | 1 | .001 | | |
| Fisher's Exact Test | | | | .001 | .000 |
| Linear-by-Linear | 13.403 | 1 | .000 | | |
| Association | | | | | |
| N of Valid Cases | 370 | | | | |

^{a}Computed only for a 2 × 2 table
^{b}0 cells (.0%) have expected count less than 5. The minimum expected count is 11.81.

TABLE 15
Summary of available peptides and responses to A*7401 patient samples

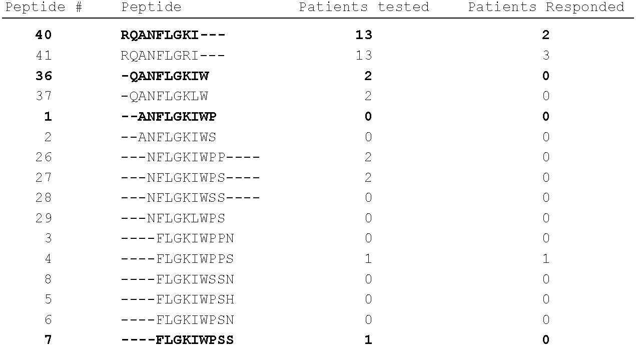

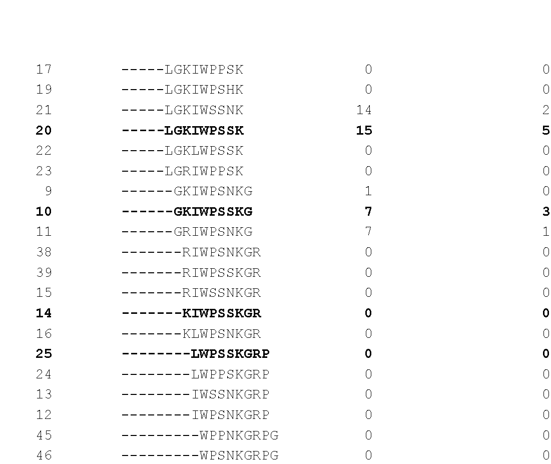

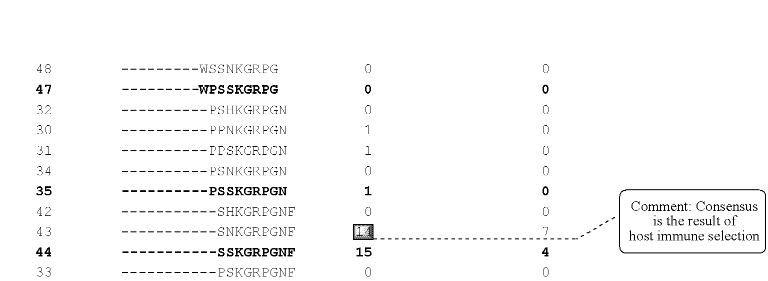

The Bolded peptides were the consensus.

TABLE 16
HLA data for all patients with A*7401 tested by ELISPOT assay

| Patient ML # | HLA-A | | HLA-B | | HLA-C | |
|---|---|---|---|---|---|---|
| 602 | 7401 | 7401 | 3501 | 3501 | 0401 | 0401 |
| 672 | 3201 | 7401 | 0705 | 5802 | 0602 | 0804 |
| 1032 | 2301 | 7401 | 3501 | 8101 | 0401 | 1801 |
| 1072 | 6802 | 7401 | 1510 | 4901 | 0304 | 0701 |
| 1076 | 6801 | 7401 | 1517 | 3501 | 0401 | 0701 |
| 1230 | 6802 | 7401 | 4703 | 5806 | 0602 | 0701 |
| 1349 | 6601 | 7401 | 3910 | 5802 | 0602 | 1701 |
| 1419 | 1010 | 7401 | 4201 | 5702 | 1701 | 1801 |
| 1481 | 0202 | 7401 | 1406 | 5001 | 0602 | 0701 |
| 1614 | 0109 | 7401 | 4415 | 1503 | 0202 | 0407 |
| 1850 | 6802 | 7401 | 4201 | 1801 | 0501 | 1701 |
| 1881 | 0301 | 7401 | 4901 | 5802 | 0602 | 0701 |
| 1901 | 0202 | 7401 | 5802 | 5802 | 0602 | 0602 |
| 1974 | 0202 | 7401 | 5802 | 5301 | 0401 | 0602 |
| 2026 | 6802 | 7401 | 1503 | 5301 | 0202 | 0415 |

TABLE 17
HLA data for patients tested by ELISPOT against
peptides GKIWPSSKG (10) or GRIWPSNKG (11)

| Pa- | | | | | | |
|---|---|---|---|---|---|---|
| tient | | | | | | |
| ML # | HLA-A | | HLA-B | | HLA-C | |
| 602¹ | [7401] | [7401] | 350101 | 350101 | 04010101 | 040101 |
| 672 | 3201 | [7401] | 0705 | 5802 | 0602 | 0804 |
| 1032¹ | 2301 | [7401] | 350101 | 8101 | 04010101 | 1801 |
| 1230¹ | 6802 | [7401] | 4703 | 5806 | 0602 | 070101 |
| 1881 | 03010101 | [7401] | 4901 | 5802 | 0602 | 070101 |
| 1901¹ | 0202 | [7401] | 5802 | 5802 | 0602 | 0602 |
| 1974 | 0202 | [7401] | 5802 | 5301 | 04010101 | 0602 |

¹[ ] did not respond

TABLE 18
HLA data for patients tested by ELISPOT against
peptides LGKIWPSSK (20) or LGKIWSSNK (21)

| Patient ML # | HLA-A | | HLA-B | | HLA-C | |
|---|---|---|---|---|---|---|
| 602¹ | [7401] | [7401] | 350101 | 350101 | 04010101 | 04010101 |
| 672¹ | 3201 | [7401] | 0705 | 5802 | 0602 | 0804 |
| 1032 | 2301 | [7401] | 350101 | 8101 | 04010101 | 1801 |
| 1072 | 6802 | [7401] | 1510 | 4901 | 030402 | 070101 |
| 1076¹ | 680101 | [7401] | 15170101 | 350101 | 04010101 | 070101 |
| 1230¹ | 6802 | [7401] | 4703 | 5806 | 0602 | 070101 |
| 1349¹ | 6601 | [7401] | 3910 | 5802 | 0602 | 1701 |
| 1419 | 010101 | [7401] | 4201 | 5702 | 1701 | 1801 |
| 1481¹ | 0202 | [7401] | 140602 | 5001 | 0602 | 070101 |
| 1614 | 0109 | [7401] | 4415 | 1503 | 020204 | 0407 |
| 1850 | 6802 | [7401] | 4201 | 180101 | 0501/03 | 1701 |
| 1881¹ | 03010101 | [7401] | 4901 | 5802 | 0602 | 070101 |
| 1901¹ | 0202 | [7401] | 5802 | 5802 | 0602 | 0602 |
| 1974 | 0202 | [7401] | 5802 | 5301 | 04010101 | 0602 |
| 2026¹ | 6802 | [7401] | 1503 | 5301 | 020204 | 0415 |

¹[ ] did not respond

TABLE 19
HLA data for patients tested by ELISPOT against
peptides RQANFLGKI (40) or RQANFLGRI (41)

| Patient ML # | HLA-A | | HLA-B | | HLA-C | |
|---|---|---|---|---|---|---|
| 602¹ | [7401] | [7401] | 350101 | 350101 | 04010101 | 04010101 |
| 672 | 3201 | [7401] | 0705 | 5802 | 0602 | 0804 |
| 1032¹ | 2301 | [7401] | 350101 | 8101 | 04010101 | 1801 |
| 1072¹ | 6802 | [7401] | 1510 | 4901 | 030402 | 070101 |
| 1076¹ | 680101 | [7401] | 15170101 | 350101 | 04010101 | 070101 |
| 1230¹ | 6802 | [7401] | 4703 | 5806 | 0602 | 070101 |
| 1481 | 0202 | [7401] | 140602 | 5001 | 0602 | 070101 |
| 1614¹ | 0109 | [7401] | 4415 | 1503 | 020204 | 0407 |
| 1850¹ | 6802 | [7401] | 4201 | 180101 | 0501/03 | 1701 |
| 1881 | 03010101 | [7401] | 4901 | 5802 | 0602 | 070101 |
| 1901¹ | 0202 | [7401] | 5802 | 5802 | 0602 | 0602 |
| 1974 | 0202 | [7401] | 5802 | 5301 | 04010101 | 0602 |
| 2026¹ | 6802 | [7401] | 1503 | 5301 | 020204 | 0415 |

¹[ ] did not respond

TABLE 20
Pooled Peptide Screen for A*7401, A*3001,
B*1402, B*4901, Cw*0602

| | | | HIV | | Positive |
|---|---|---|---|---|---|
| Allele | Mutation | Patient | status | Patient Date | Pools |
| A*7401 | K12Q/ | 1880 | pos | May 24, 2007 | 4, 7 |
| | D14E/ | 2126 | pos | Jun. 5, 2006 | 7 |
| | R20Q | | | | |
| | K387R | 2014 | pos | Jul. 22, 2003 | 40, 45 |
| | | 2026 | pos | Oct. 1, 2003 | 41 |
| | | 2215 | pos | Jul. 15, 2003 | 40, 41, 44, 45 |
| | S440N | 1227 | pos | Aug. 19, 2003 | 40, 41 |
| | | 1427 | pos | Feb. 17, 2003 | 45 |
| | | 2122 | pos | Jul. 30, 2003 | 41 |
| | | 2195 | pos | May 12, 2003 | 41 |
| | S441N | 2000 | pos | Jun. 22, 2007 | 59 |
| | | 2014 | pos | Jun. 5, 2006 | 58, 59 |
| | | 2122 | pos | Nov. 27, 2006 | 58, 59 |
| | | 1732 | neg | Nov. 16, 2006 | 58 |
| A*3001 | T115A | 2069 | pos | Jun. 6, 2003 | 26 |
| | | 2197 | pos | May 19, 2003 | 26 |
| | | 2198 | neg | May 20, 2003 | 26 |
| | K403R | 960 | pos | Jun. 13, 2005 | 57 |
| | | 1817 | neg | May 15, 2003 | 57 |
| | | 1912 | neg | Jul. 27, 2004 | 56 |
| B*1402 | S49G | 1649 | pos | May 31, 2006 | 14, 15 |
| B*4901 | T310S/ | 2142 | pos | May 23, 2006 | 30, 31, 32 |
| | E312D | 2142 | pos | Jan. 7, 2003 | 30, 31, 32 |
| | | 2244 | pos | May 16, 2006 | 30, 31, 32, 33 |
| | | 2042 | neg | Nov. 25, 2005 | 30, 31, 32 |
| Cw*0602 | K26R | 256 | pos | Jan. 29, 2007 | 8, 9 |
| | | 890 | pos | Nov. 27, 2006 | 8, 9 |
| | | 960 | pos | Nov. 15, 2006 | 8, 9 |
| | | 1250 | pos | Mar. 1, 2007 | 8, 9 |

TABLE 21
Peptide responses for A*3001, A*7401, B*1402, B*4901, Cw*0602

| | | | | | | |
|---|---|---|---|---|---|---|
| Allele | Mutation | p-value | Peptide Sequence | SFU/million | HIV status | ML |

| | | | | | | |
|---|---|---|---|---|---|---|
| A*3001 | K403R | 8.07 ×10⁻⁴ | GHIAKNCRA | 55 | negative | 1912 |
| | R20Q | 1.71 ×10⁻⁴ | IRLRPGGKK | 90 | positive | 2126 |
| | | | RLRPGGKKK | 125 | positive | 2126 |
| | K387R | 1.57 ×10⁻⁴ | QRGNFRGQK | 60 | positive | 2026 |
| | | | QRGNFRGQK | 60 | positive | 2195 |
| | | | RGNFRGQKR | 208 | positive | 2122 |
| | | | NFRGQKRIK | 55 | positive | 2014 |
| | | | RGQKRIKCF | 110 | positive | 2195 |
| | | | LRIKCFNCG | 50 | positive | 2014 |
| A*7401 | S44AN(A) | 2.46 ×10⁻⁴ | FLGKIWPSN | 55 | positive | 2215 |
| | | | GKIWPSNKG | 225 | positive | 2014 |
| | | | WPSNKGRPG | 710 | positive | 2215 |
| | | | NKGRPGNFP | 85 | positive | 2215 |
| | S441N(D) | 2.46 ×10⁻⁴ | FLGKIWPSS | 65 | positive | 2014 |
| | | | FLGKIWPSS | 150 | positive | 2122 |
| | | | KIWPSSKGR | 850 | positive | 2014 |
| | | | KIWPSSKGR | 925 | positive | 2122 |
| | | | LIWPSNKGR | 360 | positive | 2122 |
| | | | IWPSSKGRP | 115 | positive | 2014 |
| | | | IWPSSKGRP | 440 | positive | 2122 |
| | | | SSKGRPGNF | 235 | positive | 2122 |
| | | | SNKGRPGNF | 100 | positive | 2000 |
| | | | SSKGRPGNF | 120 | positive | 2014 |
| B*1402 | S49G | 2.78 ×10⁻⁴ | ERFALNPSL | 200 | positive | 1649 |
| | | | ERFALNPGL | 1210 | positive | 1649 |
| | | | RFALNPGLL | 275 | positive | 1649 |
| | | | FALNPGLLE | 105 | positive | 1649 |
| | | | LNPGLLETT | 105 | positive | 1649 |
| B*4901 | T310S/E312D | 2.47 ×10⁻⁶/ | KTLRAEQAS | 75 | positive | 2142 |
| | | 1.38 ×10⁻⁵ | LRAEQATQE | 70 | positive | 2142 |
| | | | LRAEQATQD | 85 | positive | 2142 |
| | | | LRAIQASQD | 65 | positive | 2244 |
| | | | RAEQATQEV | 1455 | positive | 2142 |
| | | | RAEQATQDV | 75 | positive | 2244 |
| | | | RAEQASQEV | 3305 | negative | 2042 |
| | | | RAEQASQEV | 1750 | positive | 2142 |
| | | | RAEQASQEV | 115 | positive | 2244 |
| | | | RAEQASQDV | 55 | positive | 2244 |
| | | | AEQATQEVK | 1020 | positive | 2042 |
| | | | AEQATQEVK | 530 | positive | 2142 |
| | | | AEQATQEVK | 55 | positive | 2244 |
| | | | AEQATQDVK | 55 | positive | 2142 |
| | | | AEQATQDVK | 70 | positive | 2244 |
| | | | AEQASQEVK | 1565 | negative | 2042 |
| | | | AEQASQEVK | 515 | positive | 2142 |
| | | | AEQASQDVK | 60 | positive | 2244 |
| | | | EQATQEVKG | 70 | positive | 2142 |
| | | | EQATQEVKG | 65 | positive | 2142 |
| | | | QATQEVKGW | 55 | positive | 2142 |
| | | | QATQEVKGW | 65 | positive | 2244 |
| | | | QASQEVKGW | 55 | positive | 2244 |
| | | | QASQDVKGW | 80 | positive | 2244 |
| | | | TQDVKGWMT | 70 | positive | 2244 |
| Cw*0602 | K26R | 8.81 × 10⁻⁵ | KIRLRPGGK | 110 | positive | 960 |
| | | | KIRLRPGGK | 50 | positive | 890 |
| | | | IRLRPGGKK | 125 | positive | 960 |
| | | | IRLRPGGKK | 125 | positive | 960 |
| | | | RLRPGGKKK | 120 | positive | 960 |
| | | | RLRPGGRKK | 85 | positive | 890 |
| | | | RLRPGGRKK | 170 | positive | 960 |
| | | | LRPGGKKKY | 398 | positive | 256 |
| | | | LRPGGKKKY | 3628 | positive | 890 |
| | | | LRPGGKKKY | 578 | positive | 1250 |
| | | | LRPGGRKKY | 343 | positive | 256 |
| | | | LRPGGRKKY | 1405 | positive | 890 |
| | | | LRPGGRKKY | 170 | positive | 1250 |
| | | | RPGGKKKYR | 50 | positive | 256 |
| | | | RPGGKKKYR | 65 | positive | 890 |
| | | | RPGGKKKYR | 55 | positive | 960 |
| | | | RPGGRKKYR | 58 | positive | 256 |
| | | | PGGRKKYRM | 110 | positive | 890 |
| | | | PGGRKKYRM | 95 | positive | 960 |
| | | | PGGRKKYRM | 153 | positive | 1250 |
| | | | GGKKKYRMK | 53 | positive | 256 |
| | | | GGKKKYRMK | 108 | positive | 1250 |
| | | | GGRKKYRMK | 60 | positive | 960 |
| | | | GRKKYRMKH | 113 | positive | 1250 |
| | | | KKKYRMKHL | 80 | positive | 960 |
| | | | RKKYRMKHL | 110 | positive | 960 |

TABLE 22

Summary of ELISPOT assay peptide responses to B*1302 patient samples

| | | |
|---|---|---|
| Patients | % responded | Mean |

| | | | | | | |
|---|---|---|---|---|---|---|
| Peptide | Re- | pep- | to | to | SFU/ | p- |

| | | | | | | | | |
|---|---|---|---|---|---|---|---|---|
| Code^{a} | Sequence | Tested^{b} | sponded | tide | either | both | million | value^{c} |

| | | | | | | | | |
|---|---|---|---|---|---|---|---|---|
| RI9c | R Q A N F L G K I - - - - - - - - - - - | 7 | 7 | 100 | 100 | 100 | 365.71 | 0.847 |
| RI9s | R Q A N F L G R I - - - - - - - - - - - | 7 | 7 | 100 | | | 406.43 | |
| QW9c | - Q A N F L G K I W - - - - - - - - - - | 5 | 0 | 0 | 0 | 0 | −48.00 | 0.298 |
| QW9s | - Q A N F L G K L W - - - - - - - - - - | 5 | 0 | 0 | | | −4.00 | |
| LK9c | - - - - - L G K I W P S S K - - - - - - | 9 | 4 | 44.4 | 44.4 | 0 | 37.22 | 0.166 |
| LK9s | - - - - - L G K I W S S N K - - - - - - | 8 | 0 | 0 | | | 6.25 | |
| GG9c | - - - - - - G K I W P S S K G - - - - - | 7 | 2 | 28.6 | 57.1 | 0 | 27.86 | 0.674 |
| GG9s | - - - - - - G R I W P S N K G - - - - - | 7 | 2 | 28.6 | | | 17.14 | |
| KR9c | - - - - - - - K I W P S S K G R - - - - | 5 | 4 | 80 | 80 | 60 | 50.00 | 0.893 |
| KR9s | - - - - - - - K L W P S N K G R - - - - | 5 | 3 | 60 | | | 55.00 | |
| SF9c | - - - - - - - - - - - S S K G R P G N F | 6 | 2 | 20 | 42.9 | 0 | 84.17 | 0.368 |
| SF9s | - - - - - - - - - - - S N K G R P G N F | 5 | 1 | 20 | | | 3.00 | |

^{a}c = consensus peptides, s = selection peptides
^{b}Patient samples were split and tested for both consensus and selection peptides
^{c}Independent samples t-test was used to compare the mean SFU/million between A*3001 and A*3002

TABLE 23
HLA data for all patients with B*1301 and B*1302
tested by ELISPOT assay

| Patient ML # | HLA-A | | HLA-B | | HLA-C | |
|---|---|---|---|---|---|---|
| 57 | 0109 | 7401 | 1301 | 3537 | 0401 | 0602 |
| 415 | 3002 | 3402 | 1302 | 5301 | 0401 | 0804 |
| 915 | 0103 | 2301 | 1302 | 5801 | 0602 | 0701 |
| 1128 | 0301 | 2902 | 1302 | 4703 | 0602 | 0701 |
| 1160 | 3402 | 3402 | 1302 | 4403 | 0401 | 0804 |
| 1720 | 2901 | 3010 | 1302 | 0705 | 0602 | 1505 |
| 1728 | 2402 | 3004 | 1302 | 5301 | 0602 | 0602 |
| 1778 | 0202 | 6802 | 1302 | 5703 | 0602 | 0701 |
| 1787 | 3001 | 6802 | 1302 | 4201 | 0602 | 1701 |
| 1937 | 2902 | 3402 | 1302 | 1503 | 0202 | 0602 |

TABLE 24

Summary of ELISPOT assay peptide responses to A*7401 patient samples

| | | |
|---|---|---|
| Patients | % responded | Mean |

| | | | | | | |
|---|---|---|---|---|---|---|
| Peptide | Re- | pep- | to | to | SFU/ | p- |

| | | | | | | | | |
|---|---|---|---|---|---|---|---|---|
| Code^{a} | Sequence | Tested^{b} | sponded | tide | either | both | million | value^{c} |

| | | | | | | | | |
|---|---|---|---|---|---|---|---|---|
| RI9c | R Q A N F L G K I - - - - - - - - - - - | 13 | 2 | 15.4 | 30.8 | 7.7 | 12.31 | 0.884 |
| RI9s | R Q A N F L G R I - - - - - - - - - - - | 13 | 3 | 23.1 | | | 15.00 | |
| LK9c | - - - - - L G K I W P S S K - - - - - - | 15 | 5 | 33.3 | 40 | 6.7 | 28.40 | 0.083 |
| LK9s | - - - - - L G K I W S S N K - - - - - - | 14 | 2 | 33.3 | | | 6.36 | |
| GG9c | - - - - - - G K I W P S S K G - - - - - | 7 | 3 | 42.9 | 42.9 | 14.3 | 68.71 | 0.571 |
| GG9s | - - - - - - G R I W P S N K G - - - - - | 7 | 1 | 14.3 | | | 39.71 | |
| SF9c | - - - - - - - - - - - S S K G R P G N F | 15 | 4 | 26.7 | 66.7 | 6.7 | 54.79 | 0.908 |
| SF9s | - - - - - - - - - - - S N K G R P G N F | 14 | 7 | 50 | | | 58.29 | |

^{a}c = consensus peptides, s = selection peptides
^{b}Patient samples were split and tested for both consensus and selection peptides
^{c}Independent samples t-test was used to compare the mean SFU/million between A*3001 and A*3002

TABLE 25

Summary of ELISPOT assay peptide responses to A*30 patient samples

| | | |
|---|---|---|
| Peptide | Patients | Mean SFU/million |

| | | | | | | |
|---|---|---|---|---|---|---|
| Code^{a} | Sequence | Tested^{b} | Responded | A*3001 | A*3002 | p-value^{c} |

| | | | | | | |
|---|---|---|---|---|---|---|
| RI9c | R Q A N F L G K I - - - - - - - - - - - | 14 | 1 | −8.33 | 26.50 | 0.052 |
| RI9s | R Q A N F L G R I - - - - - - - - - - - | 15 | 3 | 6.43 | 55.88 | 0.173 |
| NS9s | - - - N F L G K I W S S - - - - - - - - | 22 | 5 | 0.73 | 41.50 | 0.291 |
| LK9s | - - - - - L G K I W S S N K - - - - - - | 11 | 1 | 8.60 | 23.75 | 0.638 |
| LK9c | - - - - - L G K I W P S S K - - - - - - | 15 | 1 | −45.00 | −13.75 | 0.444 |
| GG9c | - - - - - - G K I W P S S K G - - - - - | 14 | 1 | −7.50 | −1.88 | 0.577 |
| GG9s | - - - - - - G R I W P S N K G - - - - - | 14 | 3 | −5.00 | 44.38 | 0.062 |
| WG9s | - - - - - - - - - W S S N K G R P G - - | 21 | 4 | 0.70 | 69.50 | 0.100 |
| WG9c | - - - - - - - - - W P S S K G R P G - - | 21 | 6 | 5.80 | 57.50 | 0.070 |
| SF9s | - - - - - - - - - - - S N K G R P G N F | 12 | 2 | 7.50 | 68.40 | 0.293 |
| SF9c | - - - - - - - - - - - S S K G R P G N F | 16 | 3 | −9.70 | 55.40 | 0.252 |

^{a}c = consensus peptides s = selection peptides
^{b}Patient samples were split and tested for both consensus and selection peptides
^{c}Independent samples t-test was used to compare the mean SFU/million between A*3001 and A*3002

TABLE 26
HLA data for all patients with A*30 tested by ELISPOT assay

| Patient ML # | HLA-A | | HLA-B | | HLA-C | |
|---|---|---|---|---|---|---|
| 79 | 3002 | 6802 | 4901 | 5802 | 0602 | 0701 |
| 199 | 3001 | 0201 | 4201 | 5703 | 0701 | 1701 |
| 273 | 3002 | 6601 | 0801 | 1405 | 0701 | 0802 |
| 415 | 3002 | 3402 | 1302 | 5301 | 0401 | 0804 |
| 752 | 3002 | 6601 | 5703 | 5802 | 0602 | 1801 |
| 810 | 3001 | 0201 | 1503 | 4201 | 0202 | 1701 |
| 890 | 3004 | 6802 | 5802 | 4503 | 0602 | 0802 |
| 960 | 3001 | 6601 | 1303 | 4415 | 0401 | 0602 |
| 1324 | 3001 | 0201 | 3501 | 5301 | 0701 | 1203 |
| 1410 | 3001 | 2301 | 1801 | 4201 | 0202 | 1701 |
| 1497 | 3002 | 0205 | 0801 | 1503 | 0401 | 0701 |
| 1530 | 3009 | 6802 | 1510 | 8101 | 0304 | 0401 |
| 1594 | 3001 | 0101 | 0801 | 4201 | 0704 | 1701 |
| 1694 | 3001 | 0201 | 5802 | 1503 | 0202 | 0602 |
| 1802 | 3002 | 0101 | 5801 | 5801 | 0302 | 0302 |
| 1843 | 3001 | 0101 | 3910 | 5702 | 1203 | 1801 |
| 1894 | 3001 | 0202 | 1503 | 5703 | 0202 | 1701 |
| 1956 | 3001 | 3002 | 4501 | 1510 | 0304 | 1601 |
| 2033 | 3002 | 3303 | 0702 | 5801 | 0302 | 0701 |
| 2052 | 3001 | 0103 | 4201 | 5802 | 0602 | 1701 |
| 2067 | 3002 | 6802 | 1510 | 3910 | 0304 | 1203 |
| 2144 | 3002 | 3402 | 1503 | 4501 | 0202 | 1601 |

We claim:

1. A peptide from the P7-P1 region of HIV-1, said P7-P1 region being defined by the sequence RQANFLGKIWPSSKGRPGNF, the peptide representing an epitope of HLA Class I B*1302 and comprising a sequence selected from the group consisting of: (i) RQANFLGKI, (ii) RQANFLGRI, (iii) KIWPSSKGR, (iv) KLWPSNKGR, (v) SNKGRPGNF, (vi) SSKGRPGNF, (vii) LGKIWPSSK and (viii) GKIWPSSKG.


SEQUENCE LISTING (205 sequences)

SEQ ID NO: 1 - protein, 20 aa, Human immunodeficiency virus
RQANFLGKIWPSSKGRPGNF

SEQ ID NO: 2 - protein, 9 aa, Human immunodeficiency virus
RQANFLGKI

SEQ ID NO: 3 - protein, 9 aa, Human immunodeficiency virus
RQANFLGRI

SEQ ID NO: 4 - protein, 9 aa, Human immunodeficiency virus
KIWPSSKGR

SEQ ID NO: 5 - protein, 9 aa, Human immunodeficiency virus
KLWPSNKGR

SEQ ID NO: 6 - protein, 9 aa, Human immunodeficiency virus
SNKGRPGNF

SEQ ID NO: 7 - protein, 9 aa, Human immunodeficiency virus
SSKGRPGNF

SEQ ID NO: 8 - protein, 9 aa, Human immunodeficiency virus
LGKIWPSSK

SEQ ID NO: 9 - protein, 9 aa, Human immunodeficiency virus
GKIWPSSKG

SEQ ID NO: 10 - protein, 9 aa, Human immunodeficiency virus
GRIWPSNKG

SEQ ID NO: 11 - protein, 9 aa, Human immunodeficiency virus
LGKIWSSNK

SEQ ID NO: 12 - protein, 9 aa, Human immunodeficiency virus
RQANFLGKI

SEQ ID NO: 13 - protein, 9 aa, Human immunodeficiency virus
KIRLRPGGK

SEQ ID NO: 14 - protein, 9 aa, Human immunodeficiency virus
KIRLRPGGK

SEQ ID NO: 15 - protein, 9 aa, Human immunodeficiency virus
GSEELRSLY

SEQ ID NO: 16 - protein, 11 aa, Human immunodeficiency virus
RSLYNTVATLY

SEQ ID NO: 17 - protein, 9 aa, Human immunodeficiency virus
ISPRTLNAW

SEQ ID NO: 18 - protein, 11 aa, Human immunodeficiency virus
KAFSPEVIPMF

SEQ ID NO: 19 - protein, 8 aa, Human immunodeficiency virus
KAFSPEVI

SEQ ID NO: 20 - protein, 10 aa, Human immunodeficiency virus
TSTLQEQIAW

SEQ ID NO: 21 - protein, 9 aa, Human immunodeficiency virus
QASQEVKNW

SEQ ID NO: 22 - protein, 9 aa, Human immunodeficiency virus
CRAPRKKGC

SEQ ID NO: 23 - protein, 10 aa, Human immunodeficiency virus
LARNCRAPRK

SEQ ID NO: 24 - protein, 10 aa, Human immunodeficiency virus
IAKNCRAPRK

SEQ ID NO: 25 - protein, 9 aa, Human immunodeficiency virus
QANFLGKIW

SEQ ID NO: 26 - protein, 9 aa, Human immunodeficiency virus
QANFLGKLW

SEQ ID NO: 27 - protein, 9 aa, Human immunodeficiency virus
SVLSGGKLD

SEQ ID NO: 28 - protein, 9 aa, Human immunodeficiency virus
SILSGGKLD

SEQ ID NO: 29 - protein, 9 aa, Human immunodeficiency virus
RASVLSGGK

SEQ ID NO: 30 - protein, 9 aa, Human immunodeficiency virus
RASVLSGGQ

SEQ ID NO: 31 - protein, 9 aa, Human immunodeficiency virus
ASVLSGGKL

SEQ ID NO: 32 - protein, 9 aa, Human immunodeficiency virus
ASVLSGGQL

SEQ ID NO: 33 - protein, 9 aa, Human immunodeficiency virus
GKLDAWEKI

SEQ ID NO: 34 - protein, 9 aa, Human immunodeficiency virus
GQLDAWEKI

SEQ ID NO: 35 - protein, 9 aa, Human immunodeficiency virus
SVLSGGKLD

SEQ ID NO: 36 - protein, 9 aa, Human immunodeficiency virus
SVLSGGKLE

SEQ ID NO: 37 - protein, 9 aa, Human immunodeficiency virus
VLSGGKLDA

SEQ ID NO: 38 - protein, 9 aa, Human immunodeficiency virus
VLSGGKLEA

SEQ ID NO: 39 - protein, 9 aa, Human immunodeficiency virus
LDAWEKIRL

SEQ ID NO: 40 - protein, 9 aa, Human immunodeficiency virus
LEAWEKIRL

SEQ ID NO: 41 - protein, 9 aa, Human immunodeficiency virus
KLDAWEKIR

SEQ ID NO: 42 - protein, 9 aa, Human immunodeficiency virus
KLDAWEKIQ

SEQ ID NO: 43 - protein, 9 aa, Human immunodeficiency virus
LDAWEKIRL

SEQ ID NO: 44 - protein, 9 aa, Human immunodeficiency virus
LEAWEKIRL

SEQ ID NO: 45 - protein, 9 aa, Human immunodeficiency virus
LDAWEKIQL

SEQ ID NO: 46 - protein, 9 aa, Human immunodeficiency virus
LEAWEKIQL

SEQ ID NO: 47 - protein, 9 aa, Human immunodeficiency virus
IRLRPGGKK

SEQ ID NO: 48 - protein, 9 aa, Human immunodeficiency virus
IQLRPGGKK

SEQ ID NO: 49 - protein, 9 aa, Human immunodeficiency virus
KIRLRPGGK

SEQ ID NO: 50 - protein, 9 aa, Human immunodeficiency virus
KIRLRPGGR

SEQ ID NO: 51 - protein, 9 aa, Human immunodeficiency virus
IRLRPGGKK

SEQ ID NO: 52 - protein, 9 aa, Human immunodeficiency virus
IRLRPGGRK

SEQ ID NO: 53 - protein, 9 aa, Human immunodeficiency virus
GKKKYRMKH

SEQ ID NO: 54 - protein, 9 aa, Human immunodeficiency virus
GRKKYRMKH

SEQ ID NO: 55 - protein, 9 aa, Human immunodeficiency virus
RMKHLVWAS

SEQ ID NO: 56 - protein, 9 aa, Human immunodeficiency virus
RMKHLVWAG

SEQ ID NO: 57 - protein, 9 aa, Human immunodeficiency virus
MKHLVWASR

SEQ ID NO: 58 - protein, 9 aa, Human immunodeficiency virus
MKHLVWAGR

SEQ ID NO: 59 - protein, 9 aa, Human immunodeficiency virus
ASRELERFA

SEQ ID NO: 60 - protein, 9 aa, Human immunodeficiency virus
AGRELERFA

SEQ ID NO: 61 - protein, 9 aa, Human immunodeficiency virus
VWASRELER

SEQ ID NO: 62 - protein, 9 aa, Human immunodeficiency virus
VWASRELEK

SEQ ID NO: 63 - protein, 9 aa, Human immunodeficiency virus
WASRELERF

SEQ ID NO: 64 - protein, 9 aa, Human immunodeficiency virus
WASRELEKF

SEQ ID NO: 65 - protein, 9 aa, Human immunodeficiency virus
ERFALNPSL

SEQ ID NO: 66 - protein, 9 aa, Human immunodeficiency virus
EKFALNPSL

SEQ ID NO: 67 - protein, 9 aa, Human immunodeficiency virus
LERFALNPS

SEQ ID NO: 68 - protein, 9 aa, Human immunodeficiency virus
LERFALNPG

SEQ ID NO: 69 - protein, 9 aa, Human immunodeficiency virus
ERFALNPSL

SEQ ID NO: 70 - protein, 9 aa, Human immunodeficiency virus
ERFALNPGL

SEQ ID NO: 71 - protein, 9 aa, Human immunodeficiency virus
PSLLETTEG

SEQ ID NO: 72 - protein, 9 aa, Human immunodeficiency virus
PGLLETTEG

SEQ ID NO: 73 - protein, 9 aa, Human immunodeficiency virus
QQIMEQLQP

SEQ ID NO: 74 - protein, 9 aa, Human immunodeficiency virus
QQIMEQLQS

SEQ ID NO: 75 - protein, 9 aa, Human immunodeficiency virus
QIMEQLQPA

SEQ ID NO: 76 - protein, 9 aa, Human immunodeficiency virus
QIMEQLQSA

SEQ ID NO: 77 - protein, 9 aa, Human immunodeficiency virus
QPALKTGTE

SEQ ID NO: 78 - protein, 9 aa, Human immunodeficiency virus
QSALKTGTE

SEQ ID NO: 79 - protein, 9 aa, Human immunodeficiency virus
QIMEQLQPA

SEQ ID NO: 80 - protein, 9 aa, Human immunodeficiency virus
QIMEQLQPS

SEQ ID NO: 81 - protein, 9 aa, Human immunodeficiency virus
IMEQLQPAL

SEQ ID NO: 82 - protein, 9 aa, Human immunodeficiency virus
IMEQLQPSL

SEQ ID NO: 83 - protein, 9 aa, Human immunodeficiency virus
PALKTGTEE

SEQ ID NO: 84 - protein, 9 aa, Human immunodeficiency virus
PSLKTGTEE

SEQ ID NO: 85 - protein, 9 aa, Human immunodeficiency virus
EELRSLFNT

SEQ ID NO: 86 - protein, 9 aa, Human immunodeficiency virus
EELRSLFNA

SEQ ID NO: 87 - protein, 9 aa, Human immunodeficiency virus
ELRSLFNTV

SEQ ID NO: 88 - protein, 9 aa, Human immunodeficiency virus
ELRSLFNAV

SEQ ID NO: 89 - protein, 9 aa, Human immunodeficiency virus
NTVATLYCV

SEQ ID NO: 90 - protein, 9 aa, Human immunodeficiency virus
NAVATLYCV

SEQ ID NO: 91 - protein, 9 aa, Human immunodeficiency virus
NTVATLYCV

SEQ ID NO: 92 - protein, 9 aa, Human immunodeficiency virus
NTVATLYCI

SEQ ID NO: 93 - protein, 9 aa, Human immunodeficiency virus
TVATLYCVH

SEQ ID NO: 94 - protein, 9 aa, Human immunodeficiency virus
TVATLYCIH

SEQ ID NO: 95 - protein, 9 aa, Human immunodeficiency virus
CVHQRIDVK

SEQ ID NO: 96 - protein, 9 aa, Human immunodeficiency virus
CIHQRIDVK

SEQ ID NO: 97 - protein, 9 aa, Human immunodeficiency virus
TLYCVHQRI

SEQ ID NO: 98 - protein, 9 aa, Human immunodeficiency virus
TLYCVHQRM

SEQ ID NO: 99 - protein, 9 aa, Human immunodeficiency virus
LYCVHQRID

SEQ ID NO: 100 - protein, 9 aa, Human immunodeficiency virus
LYCVHQRMD

SEQ ID NO: 101 - protein, 9 aa, Human immunodeficiency virus
RIDVKDTKE

SEQ ID NO: 102 - protein, 9 aa, Human immunodeficiency virus
RMDVKDTKE

SEQ ID NO: 103 - protein, 9 aa, Human immunodeficiency virus
LYCVHQRID

SEQ ID NO: 104 - protein, 9 aa, Human immunodeficiency virus
LYCVHQRIE

SEQ ID NO: 105 - protein, 9 aa, Human immunodeficiency virus
YCVHQRIDV

SEQ ID NO: 106 - protein, 9 aa, Human immunodeficiency virus
YCVHQRIEV

SEQ ID NO: 107 - protein, 9 aa, Human immunodeficiency virus
IDVKDTKEA

SEQ ID NO: 108 - protein, 9 aa, Human immunodeficiency virus
IEVKDTKEA

SEQ ID NO: 109 - protein, 9 aa, Human immunodeficiency virus
IQNKSKQKT

SEQ ID NO: 110 - protein, 9 aa, Human immunodeficiency virus
IQNKSKQKA

SEQ ID NO: 111 - protein, 9 aa, Human immunodeficiency virus
IQNKSKQKK

SEQ ID NO: 112 - protein, 9 aa, Human immunodeficiency virus
IQNKSKQKP

SEQ ID NO: 113 - protein, 9 aa, Human immunodeficiency virus
QNKSKQKTQ

SEQ ID NO: 114 - protein, 9 aa, Human immunodeficiency virus
QNKSKQKAQ

SEQ ID NO: 115 - protein, 9 aa, Human immunodeficiency virus
QNKSKQKKQ

SEQ ID NO: 116 - protein, 9 aa, Human immunodeficiency virus
QNKSKQKPQ

SEQ ID NO: 117 - protein, 9 aa, Human immunodeficiency virus
KTQQAAADT

SEQ ID NO: 118 - protein, 9 aa, Human immunodeficiency virus
KAQQAAADT

SEQ ID NO: 119 - protein, 9 aa, Human immunodeficiency virus
KKQQAAADT

SEQ ID NO: 120 - protein, 9 aa, Human immunodeficiency virus
KPQQAAADT

SEQ ID NO: 121 - protein, 9 aa, Human immunodeficiency virus
TTSTPQEQI

SEQ ID NO: 122 - protein, 9 aa, Human immunodeficiency virus
TTSTPQEQL

SEQ ID NO: 123 - protein, 9 aa, Human immunodeficiency virus
TSTPQEQIG

SEQ ID NO: 124 - protein, 9 aa, Human immunodeficiency virus
TSTPQEQLG

SEQ ID NO: 125 - protein, 9 aa, Human immunodeficiency virus
QIGWMTSNP

SEQ ID NO: 126 - protein, 9 aa, Human immunodeficiency virus
QLGWMTSNP

SEQ ID NO: 127 - protein, 9 aa, Human immunodeficiency virus
KTLRAEQAT

SEQ ID NO: 128 - protein, 9 aa, Human immunodeficiency virus
KTLRAEQAS

SEQ ID NO: 129 - protein, 9 aa, Human immunodeficiency virus
TLRAEQATQ

SEQ ID NO: 130 - protein, 9 aa, Human immunodeficiency virus
TLRAEQASQ

SEQ ID NO: 131 - protein, 9 aa, Human immunodeficiency virus
ATQEVKGWM

SEQ ID NO: 132 - protein, 9 aa, Human immunodeficiency virus
ATQDVKGWM

SEQ ID NO: 133 - protein, 9 aa, Human immunodeficiency virus
ASQEVKGWM

SEQ ID NO: 134 - protein, 9 aa, Human immunodeficiency virus
ASQDVKGWM

SEQ ID NO: 135 - protein, 9 aa, Human immunodeficiency virus
LRAEQATQE

SEQ ID NO: 136 - protein, 9 aa, Human immunodeficiency virus
LRAEQASQE

SEQ ID NO: 137 - protein, 9 aa, Human immunodeficiency virus
LRAEQATQD

SEQ ID NO: 138 - protein, 9 aa, Human immunodeficiency virus
LRAEQASQD

SEQ ID NO: 139 - protein, 9 aa, Human immunodeficiency virus
RAEQATQEV

SEQ ID NO: 140 - protein, 9 aa, Human immunodeficiency virus
RAEQATQDV

SEQ ID NO: 141 - protein, 9 aa, Human immunodeficiency virus
QEVKGWMTE

SEQ ID NO: 142 - protein, 9 aa, Human immunodeficiency virus
QDVKGWMTE

SEQ ID NO: 143 - protein, 9 aa, Human immunodeficiency virus
QEVKGWMTE

SEQ ID NO: 144 - protein, 9 aa, Human immunodeficiency virus
QEVKGWMTD

SEQ ID NO: 145 - protein, 9 aa, Human immunodeficiency virus
EVKGWMTET

SEQ ID NO: 146 - protein, 9 aa, Human immunodeficiency virus
EVKGWMTDT

SEQ ID NO: 147 - protein, 9 aa, Human immunodeficiency virus
TETLLVQNA

SEQ ID NO: 148 - protein, 9 aa, Human immunodeficiency virus
TDTLLIQNA

SEQ ID NO: 149 - protein, 9 aa, Human immunodeficiency virus
LRALGTGAT

SEQ ID NO: 150 - protein, 9 aa, Human immunodeficiency virus
LRALGTGAS

SEQ ID NO: 151 - protein, 9 aa, Human immunodeficiency virus
RALGTGATL

SEQ ID NO: 152 - protein, 9 aa, Human immunodeficiency virus
RALGTGASL

SEQ ID NO: 153 - protein, 9 aa, Human immunodeficiency virus
ATLEEMMTA

SEQ ID NO: 154 - protein, 9 aa, Human immunodeficiency virus
ASLEEMMTA

SEQ ID NO: 155 - protein, 9 aa, Human immunodeficiency virus
LKALGPAAT

SEQ ID NO: 156 - protein, 9 aa, Human immunodeficiency virus
LKALGPAAS

SEQ ID NO: 157 - protein, 9 aa, Human immunodeficiency virus
KALGPAATL

SEQ ID NO: 158 - protein, 9 aa, Human immunodeficiency virus
KALGPAASL

SEQ ID NO: 159 - protein, 9 aa, Human immunodeficiency virus
ATLEEMMTA

SEQ ID NO: 160 - protein, 9 aa, Human immunodeficiency virus
ASLEEMMTA

SEQ ID NO: 161 - protein, 9 aa, Human immunodeficiency virus
ACQGVGGPG

SEQ ID NO: 162 - protein, 9 aa, Human immunodeficiency virus
ACQGVGGPS

SEQ ID NO: 163 - protein, 9 aa, Human immunodeficiency virus
CQGVGGPGH

SEQ ID NO: 164 - protein, 9 aa, Human immunodeficiency virus
CQGVGGPSH

SEQ ID NO: 165 - protein, 9 aa, Human immunodeficiency virus
QRGNFRGQK

SEQ ID NO: 166 - protein, 9 aa, Human immunodeficiency virus
QRGNFRGQR

SEQ ID NO: 167 - protein, 9 aa, Human immunodeficiency virus
RGNFKGPKK

SEQ ID NO: 168 - protein, 9 aa, Human immunodeficiency virus
RGNFKGPRK

SEQ ID NO: 169 - protein, 9 aa, Human immunodeficiency virus
QKRIKCFNC

SEQ ID NO: 170 - protein, 9 aa, Human immunodeficiency virus
QRRIKCFNC

SEQ ID NO: 171 - protein, 9 aa, Human immunodeficiency virus
ERQANFLGK

SEQ ID NO: 172 - protein, 9 aa, Human immunodeficiency virus
ERQANFLGR

SEQ ID NO: 173 - protein, 9 aa, Human immunodeficiency virus
TAPPAESFG

SEQ ID NO: 174 - protein, 9 aa, Human immunodeficiency virus
TAPPAESFR

SEQ ID NO: 175 - protein, 9 aa, Human immunodeficiency virus
APPAESFGF

SEQ ID NO: 176 - protein, 9 aa, Human immunodeficiency virus
APPAESFRF

SEQ ID NO: 177 - protein, 9 aa, Human immunodeficiency virus
FGFGEEITS

SEQ ID NO: 178 - protein, 9 aa, Human immunodeficiency virus
FRFGEEITS

SEQ ID NO: 179 - protein, 9 aa, Human immunodeficiency virus
DKELYPLAS

SEQ ID NO: 180 - protein, 9 aa, Human immunodeficiency virus
DKELYPLAA

SEQ ID NO: 181 - protein, 9 aa, Human immunodeficiency virus
KELYPLASL

SEQ ID NO: 182 - protein, 9 aa, Human immunodeficiency virus
KELYPLAAL

SEQ ID NO: 183 - protein, 9 aa, Human immunodeficiency virus
ASLKSLFGS

SEQ ID NO: 184 - protein, 9 aa, Human immunodeficiency virus
AALKSLFGS

SEQ ID NO: 185 - protein, 9 aa, Human immunodeficiency virus
QAPPLVSLK

SEQ ID NO: 186 - protein, 9 aa, Human immunodeficiency virus
QAPPLVSLR

SEQ ID NO: 187 - protein, 9 aa, Human immunodeficiency virus
ASPLVSLKS

SEQ ID NO: 188 - protein, 9 aa, Human immunodeficiency virus
ASPLVSLRS

SEQ ID NO: 189 - protein, 9 aa, Human immunodeficiency virus
LKSLFGNDL

SEQ ID NO: 190 - protein, 9 aa, Human immunodeficiency virus
LRSLFGNDL

SEQ ID NO: 191 - protein, 9 aa, Human immunodeficiency virus
LVSLKSLFG

SEQ ID NO: 192 - protein, 9 aa, Human immunodeficiency virus
LVSLKSLFS

SEQ ID NO: 193 - protein, 9 aa, Human immunodeficiency virus
VSLKSLFGN

SEQ ID NO: 194 - protein, 9 aa, Human immunodeficiency virus
VSLKSLFSN

SEQ ID NO: 195 - protein, 9 aa, Human immunodeficiency virus
RLRPGGKKK

SEQ ID NO: 196 - protein, 9 aa, Human immunodeficiency virus
RLRPGGKKQ

SEQ ID NO: 197 - protein, 9 aa, Human immunodeficiency virus
QLRPGGKKK

SEQ ID NO: 198 - protein, 9 aa, Human immunodeficiency virus
QLRPGGKKQ

SEQ ID NO: 199 - protein, 9 aa, Human immunodeficiency virus
SLFNTVATL

SEQ ID NO: 200 - protein, 9 aa, Human immunodeficiency virus
SLYNTVATL

SEQ ID NO: 201 - protein, 9 aa, Human immunodeficiency virus
MTSNPPIPV

SEQ ID NO: 202 - protein, 9 aa, Human immunodeficiency virus
MTNNPPIPV

SEQ ID NO: 203 - protein, 10 aa, Human immunodeficiency virus
LARNCRAPRK

SEQ ID NO: 204 - protein, 10 aa, Human immunodeficiency virus
IAKNCRAPRK

SEQ ID NO: 205 - protein, 10 aa, Human immunodeficiency virus
IARNCRAPRK